SEQUENCE LISTING (3 sequences)

SEQ ID NO: 1 - protein, 1424 aa, human
MSGLGENLDPLASDSRKRKLPCDTPGQGLTCSGEKRRREQESKYIEELAELISANLSDIDNFNVKPDKCAILKETVRQIRQIKEQGKTISNDDDVQKADVSSTGQGVIDKDSLGPLLLQALDGFLFVVNRDGNIVFVSENVTQYLQYKQEDLVNTSVYNILHEEDRKDFLKNLPKSTVNGVSWTNETQRQKSHTFNCRMLMKTPHDILEDINASPEMRQRYETMQCFALSQPRAMMEEGEDLQSCMICVARRITTGERTFPSNPESFITRHDLSGKVVNIDTNSLRSSMRPGFEDIIRRCIQRFFSLNDGQSWSQKRHYQEAYLNGHAETPVYRFSLADGTIVTAQTKSKLFRNPVTNDRHGFVSTHFLQREQNGYRPNPNPVGQGIRPPMAGCNSSVGGMSMSPNQGLQMPSSRAYGLADPSTTGQMSGARYGGSSNIASLTPGPGMQSPSSYQNNNYGLNMSSPPHGSPGLAPNQQNIMISPRNRGSPKIASHQFSPVAGVHSPMASSGNTGNHSFSSSSLSALQAISEGVGTSLLSTLSSPGPKLDNSPNMNITQPSKVSNQDSKSPLGFYCDQNPVESSMCQSNSRDHLSDKESKESSVEGAENQRGPLESKGHKKLLQLLTCSSDDRGHSSLTNSPLDSSCKESSVSVTSPSGVSSSTSGGVSSTSNMHGSLLQEKHRILHKLLQNGNSPAEVAKITAEATGKDTSSITSCGDGNVVKQEQLSPKKKENNALLRYLLDRDDPSDALSKELQPQVEGVDNKMSQCTSSTIPSSSQEKDPKIKTETSEEGSGDLDNLDAILGDLTSSDFYNNSISSNGSHLGTKQQVFQGTNSLGLKSSQSVQSIRPPYNRAVSLDSPVSVGSSPPVKNISAFPMLPKQPMLGGNPRMMDSQENYGSSMGGPNRNVTVTQTPSSGDWGLPNSKAGRMEPMNSNSMGRPGGDYNTSLPRPALGGSIPTLPLRSNSIPGARPVLQQQQQMLQMRPGEIPMGMGANPYGQAAASNQLGSWPDGMLSMEQVSHGTQNRPLLRNSLDDLVGPPSNLEGQSDERALLDQLHTLLSNTDATGLEEIDRALGIPELVNQGQALEPKQDAFQGQEAAVMMDQKAGLYGQTYPAQGPPMQGGFHLQGQSPSFNSMMNQMNQQGNFPLQGMHPRANIMRPRTNTPKQLRMQLQQRLQGQQFLNQSRQALELKMENPTAGGAAVMRPMMQPQVSSQQGFLNAQMVAQRSRELLSHHFRQQRVAMMMQQQQQQQQQQQQQQQQQQQQQQQQQQQQQTQAFSPPPNVTASPSMDGLLAGPTMPQAPPQQFPYQPNYGMGQQPDPAFGRVSSPPNAMMSSRMGPSQNPMMQHPQAASIYQSSEMKGWPSGNLARNSSFSQQQFAHQGNPAVYSMVHMNGSSGHMGQMNMNPMPMSGMPMGPDQKYC

SEQ ID NO: 2 - DNA, 6845 bp, Human
cggcagcggctgcggcttagtcggtggcggccggcggcggctgcgggctgagcggcgagtttccgatttaaagctgagctgcgaggaaaatggcggcgggaggatcaaaatacttgctggatggtggactcagagaccaataaaaataaactgcttgaacatcctttgactggttagccagttgctgatgtatattcaagatgagtggattaggagaaaacttggatccactggccagtgattcacgaaaacgcaaattgccatgtgatactccaggacaaggtcttacctgcagtggtgaaaaacggagacgggagcaggaaagtaaatatattgaagaattggctgagctgatatctgccaatcttagtgatattgacaatttcaatgtcaaaccagataaatgtgcgattttaaaggaaacagtaagacagatacgtcaaataaaagagcaaggaaaaactatttccaatgatgatgatgttcaaaaagccgatgtatcttctacagggcagggagttattgataaagactccttaggaccgcttttacttcaggcattggatggtttcctatttgtggtgaatcgagacggaaacattgtatttgtatcagaaaatgtcacacaatacctgcaatataagcaagaggacctggttaacacaagtgtttacaatatcttacatgaagaagacagaaaggattttcttaagaatttaccaaaatctacagttaatggagtttcctggacaaatgagacccaaagacaaaaaagccatacatttaattgccgtatgttgatgaaaacaccacatgatattctggaagacataaacgccagtcctgaaatgcgccagagatatgaaacaatgcagtgctttgccctgtctcagccacgagctatgatggaggaaggggaagatttgcaatcttgtatgatctgtgtggcacgccgcattactacaggagaaagaacatttccatcaaaccctgagagctttattaccagacatgatctttcaggaaaggttgtcaatatagatacaaattcactgagatcctccatgaggcctggctttgaagatataatccgaaggtgtattcagagattttttagtctaaatgatgggcagtcatggtcccagaaacgtcactatcaagaagcttatcttaatggccatgcagaaaccccagtatatcgattctcgttggctgatggaactatagtgactgcacagacaaaaagcaaactcttccgaaatcctgtaacaaatgatcgacatggctttgtctcaacccacttccttcagagagaacagaatggatatagaccaaacccaaatcctgttggacaagggattagaccacctatggctggatgcaacagttcggtaggcggcatgagtatgtcgccaaaccaaggcttacagatgccgagcagcagggcctatggcttggcagaccctagcaccacagggcagatgagtggagctaggtatgggggttccagtaacatagcttcattgacccctgggccaggcatgcaatcaccatcttcctaccagaacaacaactatgggctcaacatgagtagccccccacatgggagtcctggtcttgccccaaaccagcagaatatcatgatttctcctcgtaatcgtgggagtccaaagatagcctcacatcagttttctcctgttgcaggtgtgcactctcccatggcatcttctggcaatactgggaaccacagcttttccagcagctctctcagtgccctgcaagccatcagtgaaggtgtggggacttcccttttatctactctgtcatcaccaggccccaaattggataactctcccaatatgaatattacccaaccaagtaaagtaagcaatcaggattccaagagtcctctgggcttttattgcgaccaaaatccagtggagagttcaatgtgtcagtcaaatagcagagatcacctcagtgacaaagaaagtaaggagagcagtgttgagggggcagagaatcaaaggggtcctttggaaagcaaaggtcataaaaaattactgcagttacttacctgttcttctgatgaccggggtcattcctccttgaccaactcccccctagattcaagttgtaaagaatcttctgttagtgtcaccagcccctctggagtctcctcctctacatctggaggagtatcctctacatccaatatgcatgggtcactgttacaagagaagcaccggattttgcacaagttgctgcagaatgggaattcaccagctgaggtagccaagattactgcagaagccactgggaaagacaccagcagtataacttcttgtggggacggaaatgttgtcaagcaggagcagctaagtcctaagaagaaggagaataatgcacttcttagatacctgctggacagggatgatcctagtgatgcactctctaaagaactacagccccaagtggaaggagtggataataaaatgagtcagtgcaccagctccaccattcctagctcaagtcaagagaaagaccctaaaattaagacagagacaagtgaagagggatctggagacttggataatctagatgctattcttggtgatctgactagttctgacttttacaataattccatatcctcaaatggtagtcatctggggactaagcaacaggtgtttcaaggaactaattctctgggtttgaaaagttcacagtctgtgcagtctattcgtcctccatataaccgagcagtgtctctggatagccctgtttctgttggctcaagtcctccagtaaaaaatatcagtgctttccccatgttaccaaagcaacccatgttgggtgggaatccaagaatgatggatagtcaggaaaattatggctcaagtatgggtgggccaaaccgaaatgtgactgtgactcagactccttcctcaggagactggggcttaccaaactcaaaggccggcagaatggaacctatgaattcaaactccatgggaagaccaggaggagattataatacttctttacccagacctgcactgggtggctctattcccacattgcctcttcggtctaatagcataccaggtgcgagaccagtattgcaacagcagcagcagatgcttcaaatgaggcctggtgaaatccccatgggaatgggggctaatccctatggccaagcagcagcatctaaccaactgggttcctggcccgatggcatgttgtccatggaacaagtttctcatggcactcaaaataggcctcttcttaggaattccctggatgatcttgttgggccaccttccaacctggaaggccagagtgacgaaagagcattattggaccagctgcacactcttctcagcaacacagatgccacaggcctggaagaaattgacagagctttgggcattcctgaacttgtcaatcagggacaggcattagagcccaaacaggatgctttccaaggccaagaagcagcagtaatgatggatcagaaggcaggattatatggacagacatacccagcacaggggcctccaatgcaaggaggctttcatcttcagggacaatcaccatcttttaactctatgatgaatcagatgaaccagcaaggcaattttcctctccaaggaatgcacccacgagccaacatcatgagaccccggacaaacacccccaagcaacttagaatgcagcttcagcagaggctgcagggccagcagtttttgaatcagagccgacaggcacttgaattgaaaatggaaaaccctactgctggtggtgctgcggtgatgaggcctatgatgcagccccaggtgagctcccagcagggttttcttaatgctcaaatggtcgcccaacgcagcagagagctgctaagtcatcacttccgacaacagagggtggctatgatgatgcagcagcagcagcagcagcaacagcagcagcagcagcagcagcagcagcaacagcaacagcaacagcaacagcagcaacagcagcaaacccaggccttcagcccacctcctaatgtgactgcttcccccagcatggatgggcttttggcaggacccacaatgccacaagctcctccgcaacagtttccatatcaaccaaattatggaatgggacaacaaccagatccagcctttggtcgagtgtctagtcctcccaatgcaatgatgtcgtcaagaatgggtccctcccagaatcccatgatgcaacacccgcaggctgcatccatctatcagtcctcagaaatgaagggctggccatcaggaaatttggccaggaacagctccttttcccagcagcagtttgcccaccaggggaatcctgcagtgtatagtatggtgcacatgaatggcagcagtggtcacatgggacagatgaacatgaaccccatgcccatgtctggcatgcctatgggtcctgatcagaaatactgctgacatctctgcaccaggacctcttaaggaaaccactgtacaaatgacactgcactaggattattgggaaggaatcattgttccaggcatccatcttggaagaaaggaccagctttgagctccatcaagggtattttaagtgatgtcatttgagcaggactggattttaagccgaagggcaatatctacgtgtttttcccccctccttctgctgtgtatcatggtgttcaaaacagaaatgttttttggcattccacctcctagggatataattctggagacatggagtgttactgatcataaaacttttgtgtcacttttttctgccttgctagccaaaatctcttaaatacacgtaggtgggccagagaacattggaagaatcaagagagattagaatatctggtttctctagttgcagtattggacaaagagcatagtcccagccttcaggtgtagtagttctgtgttgaccctttgtccagtggaattggtgattctgaattgtcctttactaatggtgttgagttgctctgtccctattatttgccctaggctttctcctaatgaaggttttcatttgccattcatgtcctgtaatacttcacctccaggaactgtcatggatgtccaaatggctttgcagaaaggaaatgagatgacagtatttaatcgcagcagtagcaaacttttcacatgctaatgtgcagctgagtgcactttatttaaaaagaatggataaatgcaatattcttgaggtcttgagggaatagtgaaacacattcctggtttttgcctacacttacgtgttagacaagaactatgatttttttttttaaagtactggtgtcaccctttgcctatatggtagagcaataatgctttttaaaaataaacttctgaaaacccaaggccaggtactgcattctgaatcagaatctcgcagtgtttctgtgaatagatttttttgtaaatatgacctttaagatattgtattatgtaaaatatgtatatacctttttttgtaggtcacaacaactcatttttacagagtttgtgaagctaaatatttaacattgttgatttcagtaagctgtgtggtgaggctaccagtggaagagacatcccttgacttttgtggcctgggggaggggtagtgctccacagcttttccttccccaccccccagccttagatgcctcgctcttttcaatctcttaatctaaatgctttttaaagagattatttgtttagatgtaggcattttaattttttaaaaattcctctaccagaactaagcactttgttaatttggggggaaagaatagatatggggaaataaacttaaaaaaaaatcaggaatttaaaaaaacgagcaatttgaagagaatcttttggattttaagcagtccgaaataatagcaattcatgggctgtgtgtgtgtgtgtatgtgtgtgtgtgtgtgtgtatgtttaattatgttaccttttcatcccctttaggagcgttttcagattttggttgctaagacctgaatcccatattgagatctcgagtagaatccttggtgtggtttctggtgtctgctcagctgtcccctcattctactaatgtgatgctttcattatgtccctgtggattagaatagtgtcagttatttcttaagtaactcagtacccagaacagccagttttactgtgattcagagccacagtctaactgagcaccttttaaacccctccctcttctgccccctaccacttttctgctgttgcctctctttgacacctgttttagtcagttgggaggaagggaaaaatcaagtttaattccctttatctgggttaattcatttggttcaaatagttgacggaattgggtttctgaatgtctgtgaatttcagaggtctctgctagccttggtatcattttctagcaataactgagagccagttaattttaagaatttcacacatttagccaatctttctagatgtctctgaaggtaagatcatttaatatctttgatatgcttacgagtaagtgaatcctgattatttccagacccaccaccagagtggatcttattttcaaagcagtatagacaattatgagtttgccctctttcccctaccaagttcaaaatatatctaagaaagattgtaaatccgaaaacttccattgtagtggcctgtgcttttcagatagtatactctcctgtttggagacagaggaagaaccaggtcagtctgtctctttttcagctcaattgtatctgacccttctttaagttatgtgtgtggggagaaatagaatggtgctcttatctttcttgactttaaaaaaattattaaaaacaaaaaaaaaataaa

SEQ ID NO: 3 - protein, 1255 aa, Human [sequence omitted]

(19) United States
(12) Patent Application Publication
(10) Pub. No.: US 2003/0224467 A1
(43) Pub. Date: Dec. 4, 2003

(54) AIB1 as a prognostic marker and predictor of resistance to endocrine therapy

(76) Inventors: C. Kent Osborne, Houston, TX (US); Rachel Schiff, Houston, TX (US); Valerie Bardou, Marseille (FR); Susan Hilsenbeck, Houston, TX (US); Gary Clark, Houston, TX (US); Jiemin Wong, Pearland, TX (US); Gary Chamness, Houston, TX (US); Torsten Hopp, Pearland, TX (US)

Correspondence Address: FULBRIGHT & JAWORSKI, LLP, 1301 MCKINNEY, SUITE 5100, HOUSTON, TX, 77010-3095, US

(21) Appl. No.: 10/418,027

(22) Filed: Apr. 17, 2003

Related U.S. Application Data
(60) Provisional application No. 60/373,237, filed on Apr. 17, 2002.

(51) Int. Cl. C12Q 1/68; G01N 33/574

(52) U.S. Cl. 435/7.23; 435/6

(57) ABSTRACT

The present invention is directed to methods involving an AIB1 polypeptide. More particularly, the methods are directed to identifying an endocrine therapy-resistant patient, to providing a prognosis for disease-free survival of a cancer patient and to treating a cancer patient that is endocrine therapy-resistant.

### CROSS-REFERENCE TO RELATED APPLICATIONS

This application claims the benefit of U.S. Provisional Application No. 60/373,237, filed Apr. 17, 2002, which is hereby incorporated by reference in its entirety.

### STATEMENT REGARDING FEDERALLY SPONSORED RESEARCH OR DEVELOPMENT

The present invention was developed with funds from the United States Government grant number CA30195 and CA50183. Therefore, the United States Government may have certain rights in the invention.

### TECHNICAL FIELD

The present invention relates to the fields of biotechnology, cell biology and medicine. More specifically, the present invention relates to methods of providing a prognosis of disease-free survival for a cancer patient. The present invention also relates to methods of predicting endocrine therapy resistance., and to treatments for patients receiving endocrine therapy.

### BACKGROUND OF THE INVENTION

Steroids, thyroid hormones, vitamin D and retinoids regulate diverse biological processes including growth, development, and homeostasis and particularly play major roles in the development of the normal mammary gland and in breast tumor development through their cognate nuclear hormone receptors which make up a superfamily of structurally related intracellular ligand-activated transcription factors (Horwitz et al., 1996; Shibata et al., 1997; Tsai et al., 1994). Estrogen receptors (ER) are members of a structurally related ligand-activated transcription factor superfamily.

An ER mediates the effects of estrogen on the development and progression of breast cancer, and it serves as an important diagnostic and therapeutic target. ER regulates the expression of genes which contribute to tumor progression by classical mechanisms such as binding to specific response elements in a gene promoter and non-classical mechanisms to activate growth factor pathways at the membrane. The drug tamoxifen partially inhibits ER activity and is an effective treatment of breast cancer.

ER-mediated gene transcription is regulated not only by ER binding of estrogens or anti-estrogens, but also by other post-translational events such as receptor phosphorylation, which is induced by ligand binding and by mitogen-activated protein kinase (MAPK) pathways (Ali et al., 1993; Arnold et al., 1994; Le Goff et al., 1994; Kato et al., 1995; Bunone et al., 1996; Pietras et al., 1995). ER function is also modulated by the interaction of a receptor protein with coregulatory proteins that serve either as coactivators to enhance gene transcription or as corepressors to inhibit the ER (McKenna et al., 1999). These coregulatory molecules also alter ER activity when receptor bound to selective ER modulators such as tamoxifen, a drug with mixed estrogen agonist/antagonist properties. In cultured cells in which the levels of these proteins can be dramatically manipulated (Jackson et al., 1997; McInerney et al., 1996; Lavinsky et al., 1998; Shiau et al., 1998; and Smith et al, 1997), increased coactivator levels enhance the agonist activity of tamoxifen-bound ER, and corepressors enhance antagonist activity.

AIB1, also called SRC-3, RAC3, ACTR, or p/CIP, is an ER coactivator that has been directly linked to breast cancer (Anzick et al., 1997; Torchia et al., 1997; Li et al., 1997; and Chen et al., 1997). It is overexpressed in breast cancer as compared to normal duct epithelium, and it is amplified in a small proportion of tumors(Anzick et al., 1997; Murphy et al., 2000; Bautista et al., 1998; and Bouras et al., 2001). AIB1 is highly expressed in cultured MCF-7 human breast cancer cells, and its biological activity is crucial for growth of these cells in vitro and in vivo (List et al., 2001). AIB1 is phosphorylated by MAPKs. AIB1 has also been indicated as an important modifier gene contributing to the high incidence of breast cancer in patients carrying BRCA-1 mutations (Rebbeck, et al., 2001).

WO 98/57982 to Meltzker describes a polynucleotide sequence encoding AIB1 which is over-expressed in breast cancer cells, diagnostic assays for tamoxifen-sensitivity and screening assays to identify inhibitors of the interaction between AIB1 and the steroid hormone receptor (e.g., ER). Meltzker teaches that the absence of AIB1 , e.g., loss of the AIB1 gene, but retention of ER-positivity in steroid hormone-responsive cancers predicts failure or poor responsiveness to anti-endocrine therapy, e.g., administration of anti-estrogen compositions such as tamoxifen. However, this teaching is in sharp contrast to the present invention. As is described herein resistance to endocrine therapies such as tamoxifen is predicted in cancer patients by observing a high AIB1 polypeptide level.

Only a few studies have investigated the expression of ER coactivators in clinical breast cancer samples, and only one of these employed protein measurements rather than mRNA (List et al., 2001). This study showed that AIB1 protein is expressed at increased levels in tumors compared to normal breast. AIB1 is gene-amplified in a small proportion of ER positive breast cancers, and it also appears to be overexpressed in 30-60% of breast cancers compared to normal breast epithelium by mRNA in situ hybridization (Anzick et al., 1997; Murphy et al., 2000; Bautista et al., 1998; and Bouras et al., 2001). One study found a correlation between ER and AIB1 (Bautista et al., 1998). Another study measuring AIB1 mRNA by in situ hybridization reported that expression was higher in high grade tumors, in tumors that were negative for ER and PR, and in tumors that showed intense staining for p53 and HER-2 (Bouras et al., 2001).

There are no prior studies correlating AIB1 with clinical outcome. One study of 21 primary breast tumors measured mRNA levels of SRC 1, an ER coactivator related to AIB1 (Berns et al., 1998). SRC1 expression was reduced in tumors as compared to normal ductal epithelium, and high levels of SRC1 in the primary tumor correlated with response to tamoxifen given at the time of distant recurrence.

Prior to the present invention, laboratory studies indicated that ER-positive breast cancers which overexpress HER-2 may be relatively resistant to tamoxifen (Benz et al., 1993). The mechanisms for this resistance are not yet clear, although ligand-independent activation of the ER by activated MAPKs in such tumors are expected to contribute (Kato, 1995; Bunone et al., 1996; Pietras et al., 1995). High HER-2 expression has also been shown to correlate with tamoxifen resistance in patients in some studies, but this association is not strong and other studies have failed to confirm the association (Mass, R., 2000; Ciocca and Elledge, 2000).

The cumulative data indicate that ER coregulators are important contributors to estrogen-mediated tumor growth and, potentially, to sensitivity or resistance to endocrine therapy. Tumors with a relative abundance of coactivators are hypothesized to be less responsive to tamoxifen therapy because of increased estrogen agonist activity of tamoxifen-bound ER (Smith et al., 1997; and Takimoto, G. S., 1999). However, the lack of clinical investigations has hindered further development of this hypothesis.

The present invention provides long-sought clinical data and observations of cancer patients having tumors expressing ER coactivators. In particular, the coactivator AIB1 demonstrated a surprising and significant relationship with tamoxifen effectiveness. The identified relationship led to the development of novel methods to predict disease-free survival of a cancer patient, in particular hormone-responsive cancers. The invention establishes that the ER coactivator AIB1 is an important molecular marker for determining disease-free survival, for identifying endocrine therapy-resistant patients and for treating those patients. Specifically, AIB1 gene expression levels serve as a predictor of endocrine therapy resistance and as an advers prognostic marker. In contrast, high AIB 1 polypeptide levels predict favorable disease-free survival in patients not receiving endocrine therapy, and serve as a prognostic marker in patients receiving endocrine therapy. Further, the predictive significance of the biomarker combination of AIB1 and HER-2 is also described.

### BRIEF SUMMARY OF THE INVENTION

The invention provides a method of identifying patient with endocrine therapy-resistant cancer comprising obtaining a sample from the patient; and determining an AIB1 polypeptide level, wherein an elevated AIB1 polypeptide level as compared to a control sample indicates that the patient is endocrine therapy-resistant. In one embodiment of the invention, the sample is a fluid, a tissue or a cell. In another embodiment of the invention, the AIB1 polypeptide comprises an amino acid sequence substantially similar to SEQ ID NO:1. In another specific embodiment, the AIB1 polypeptide is an alternatively spliced form of AIB1. In a specific embodiment of the invention, the cancer comprises an estrogen receptor-positive cancer or a progesterone receptor-positive cancer. In a specific embodiment of the invention, the cancer is a breast an ovarian, a prostate, or an endometrial cancer. A specific embodiment of the invention further comprises the step of measuring a HER-2 polypeptide level, wherein an elevated HER-2 polypeptide level together with the elevated AIB1 polypeptide level as compared to the control indicates that the patient is endocrine therapy-resistant. It is contemplated in a specific embodiment that the AIB1 polypeptide comprises an amino acid sequence substantially similar to SEQ ID NO:1. The HER-2 polypeptide comprises an amino acid sequence substantially similar to SEQ ID NO:3. It is contemplated in a specific embodiment that the HER-2 polypeptide comprises an amino acid sequence substantially similar to SEQ ID NO:3.

Provided in the present invention is a method of providing a prognosis of disease-free survival in a cancer patient comprising the steps of obtaining a sample from the patient; and determining an AIB1 polypeptide level in the sample, wherein an elevated AIB1 polypeptide level as compared to a control sample indicates the good prognosis of a prolonged disease-free survival. In a specific embodiment of the invention, the patient is not receiving an endocrine therapy, a chemotherapy or a hormonal therapy. In another embodiment, the patient is concurrently receiving an endocrine therapy, a chemotherapy or a hormonal therapy. In a specific embodiment, the method further comprises determining a HER-2 polypeptide level in the sample, wherein an elevated AIB1 polypeptide level and an elevated HER-2 polypeptide level as compared to a control sample in a patient concurrently receiving an endocrine therapy, a chemotherapy or a hormonal therapy indicates the prognosis of a low disease-free survival. In a specific embodiment, the endocrine therapy comprises tamoxifen, raloxifene, megestrol, or toremifene. In a further specific embodiment, the aromatase inhibitor is anastrozole, letrozole, or exemestane, or pure anti-estrogens such fulvestrant, or surgical or medical means (goserelin, leuprolide) for reducing ovarian function. In a further specific embodiment, the cancer comprises an estrogen receptor-positive cancer or a progesterone receptor-positive cancer.

Provided in the present invention is a method of providing a treatment decision for a cancer patient receiving an endocrine therapy comprising obtaining a sample from the patient; and comprising the steps of obtaining a sample from the patient; and determining an AIB1 polypeptide level in the sample, wherein an elevated AIB1 polypeptide level as compared to a control sample indicates that that cancer is endocrine therapy resistant.

An embodiment of the invention is a method of screening for a compound that improves the effectiveness of an endocrine therapy in a patient comprising the steps of: introducing to a cell a test agent, wherein the cell comprises a polynucleotide encoding a AIB1 polypeptide under control of a promoter operable in the cell; and measuring the AIB1 polypeptide level, wherein when the level is decreased following the introduction, the test agent is the compound that improves effectiveness of the endocrine therapy in the patient. It is also contemplated that such an agent will prevent the development of endocrine therapy resistance in a patient receiving such a therapy. In a specific embodiment, the patient is endocrine therapy-resistant. In a further specific embodiment, the endocrine therapy comprises an adjuvant. It is also contemplated that the compound is a ribozyme, an antisense nucleotide, a receptor blocknig antibody, a small molecule inhibitor, or a promoter inhibitor.

An embodiment of the invention is a method of screening for a compound that improves the effectiveness of an endocrine therapy in a patient comprising the steps of: contacting a test agent with an AIB1 polypeptide or an estrogen receptor (ER) polypeptide, wherein the AIB1 polypeptide or the ER polypeptide is linked to a marker; and determining the ability of the test agent to interfere with the binding of the AIB1 polypeptide and the ER polypeptide, wherein when the marker level is decreased following the contacting, the test agent is the compound that improves effectiveness of the endocrine therapy in the patient. In certain embodiments of the invention, the patient is endocrine therapy-resistant.

One embodiment of the invention is a method of treating a cancer patient comprising administering to the patient a therapeutically effective amount of an antagonist of an AIB1 polypeptide and an endocrine therapy. In certain embodiments of the invention, the patient is endocrine therapy-resistant. A specific embodiment of the invention is presented wherein the antagonist interferes with translation of the AIB1 polypeptide. In a further specific embodiment of the invention the antagonist interferes with an interaction between the AIB1 polypeptide and an estrogen receptor polypeptide. The antagonist interferes with phosphorylation or any other posttranslational modification of the AIB1 polypeptide in yet another specific embodiment of the invention. In another specific embodiment of the invention, the antagonist inhibits the function of a polypeptide encoding a kinase that specifically phosphorylates the AIB1 polypeptide. In another embodiment, the antagonist is administered before, together with, or after the endocrine therapy. The antagonist and the endocrine therapy are administered at the same time in another embodiment.

An embodiment of the invention is method of improving the effectiveness of an endocrine therapy in a cancer patient comprising administering a therapeutically effective amount of an antagonist of an AIB1 polypeptide level to the patient to provide a therapeutic benefit to the patient. In a specific embodiment, the administering is systemic, regional, local or direct with respect to the cancer.

An embodiment of the invention is a method of treating a cancer patient comprising: identifying an antagonist of an AIB1 polypeptide by introducing to a cell a test agent, wherein the cell comprises a polynucleotide encoding a AIB1 polypeptide under control of a promoter operable in the cell, and measuring the AIB1 polypeptide level, wherein when the level is decreased following the introduction, the test agent is the antagonist of the AIB1 polypeptide; and administering to the patient a therapeutically effective amount of the antagonist. In certain embodiments of the invention, the patient is endocrine therapy-resistant.

An embodiment of the invention is a method of determining whether a pre-menopausal breast cancer patient should have ovariectomy as a treatment option (also goserulin, leupitine, letrozole, exesmestane, anastrozole, fulvestrant). Elevated levels of AIB1 and HER-2 in a tumor sample are indicative of ovariectomy as a possible treatment option.

An embodiment of the invention is a method of determining whether a cancer patient has de novo endocrine therapy resistance comprising the steps of:obtaining a sample from the patient; and determining an AIB1 polypeptide level in the sample and a HER-2 polypeptide level in the sample, wherein an elevated AIB1 polypeptide level and an elevated HER-2 polypeptide level as compared to a control sample indicate de novo endocrine therapy resistance.

Other embodiments, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF SUMMARY OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1. shows AIB1 Western blots from tumor extracts.

FIG. 2. is a graph of disease-free survival as a function of AIB1 expression levels in patients not receiving adjuvant therapy.

FIG. 3. is a graph of disease-free survival as a function of AIB1 and HER-2 expression levels in patients not receiving adjuvant therapy.

FIG. 4. is a graph of disease-free survival as a function of AIB1 and HER-2 expression levels in patients receiving adjuvant therapy.

FIG. 5. illustrates that HER-2 overexpression results in tamoxifen-stimulated growth of breast cancer cells. FIG. 5A shows that treatment of MCF-7/HER-218 tumors in mice with estrogen or tamoxifen caused increased tumor growth. FIG. 5B shows that both estrogen and tamoxifen activate MAPK in these tumors. AIB1 expression and phosphorylation are induced by both tamoxifen and estrogen FIG. 5D indicates the results of an anchorage independent growth assay comparing MCF-7 and MCF-7/HER-218. Only in the presence of high HER-2+AIB1 estrogen and tamoxifen are agonists. FIG. 5E. shows the percentage of MCF-7 and MCF-7/HER-218 cells in S-phase. Elevated HER-2 levels induce S-phase in MCF-7 HER-218 cells, which are HER-2 overexpressing cells.

FIG. 6. illustrates the cross talk between growth factor receptor and ER pathways. FIG. 6A and B shows that phosphorylated Her-2/neu and the downstream kinases p42-44 MAPK (MAP kinase) and Akt were increased by both estrogen and tamoxifen only in cells that overexpress HER-2, namely MCF-7 HER-2-18 and BT474-HER-2. FIG. 6C shows the effects in FIG. 6A are evident after 48 hour treatment instead of 20 minute treatment with estrogen and tamoxifen.

FIG. 7. show phosphorylation of AIB1 by heregulin, estrogen and tamoxifen.

FIG. 8. shows that in the presence of elevated HER-2 tamoxifen is an agonist of ER dependent gene transcription. FIG. 8A shows the ER-dependent gene transcription in the context of tamoxifen, estrogen, and heregulin. FIG. 8B shows the agonist properties of tamoxifen in on endogenous genes in HER-2/neu-overexpressing cells. FIG. 8C illustrates the results of chromatin immunoprecipitation experiments on the pS2 promoter in MCF-7 and MCF-7/HER-218 cells, where AIB1 is recruited only the presence of elevated HER-2, like in MCF-HER-2

FIG. 9 shows tumor volumes in athymic mice with MCF-7/HER-218 tumors with and without estrogen deprivation and with and without Herceptin, which blocks the HER-2 pathway.

FIG. 10 shows a schematic of HER-2 signaling.

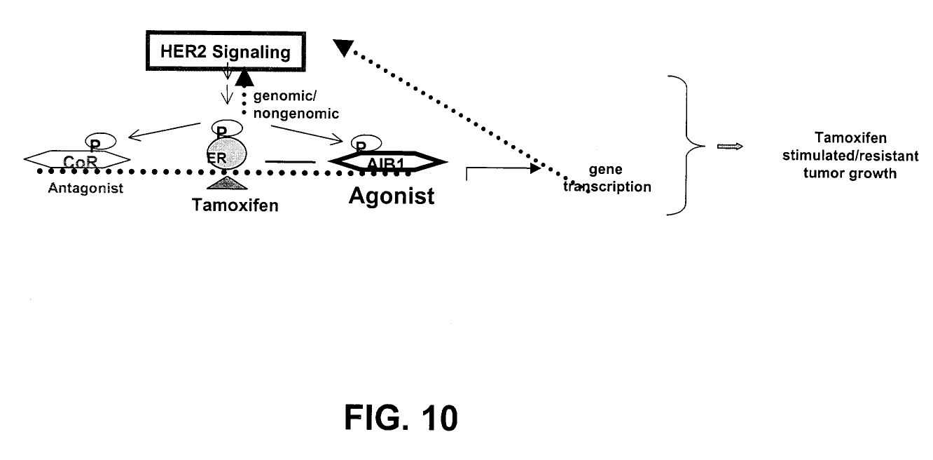

### DETAILED DESCRIPTION OF THE INVENTION

1. Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

As used herein, the term “adjuvant” refers to a pharmacological agent that is provided to a patient as an additional therapy to the primary treatment of a disease or condition.

The term “antagonist” as used herein is defined as a factor which interferes with, neutralizes or impedes the activity, function or effect of another biological entity. The agent may partially or completely interfere with a biological activity. An antagonist of AIB1 may interfere with the activity of AIB1, or the number of AIB1 polypeptides in a cell. Thus, an antagonist of AIB1 may be a compound that interferes with posttranslational modifications of AIB1. It may be an antisense molecule that interferes with the translation of AIB1. An antagonist of AIB1 may be a specific protease that decreases the number of AIB1 polypeptides in a cell. An antagonist of AIB1 may be a promoter downregulator that decreases the levels of AIB1 transcripts. An antagonist of AIB1 may also be a downregulator of AIB1.

The term “interfere with binding of AIB1” as used herein refers to interfering with either physiological binding, e.g. providing a physical barrier to binding, or functional binding of AIB1. Inhibition of posttranslational modifications of AIB1, such as phosphorylation or methylation, may form functional barriers to binding.

The term “interaction of AIB1 polypeptide and estrogen receptor polypeptide” refers to an interaction that is functional or productive. Such an interaction may lead to downstream signaling events. Other contemplated interactions allow further productive binding events with other molecules.

The term “control sample” as used herein indicates a sample that is compared to a patient sample. A control sample may be obtained from the same tissue that the patient sample is taken from. However, a noncancerous area may be chosen to reflect the AIB1 or HER-2 polypeptide levels in normal cells for a particular patient. A control may be a cell line, such as MCF-7, in which serial dilutions are undertaken to determine the exact concentration of elevated AIB1 polypeptide levels. Such levels are compared with a patient sample. A “control sample” may comprise a theoretical patient with an elevated AIB1 or HER-2 polypeptide level that is calculated to be the cutoff point for elevated AIB1 or HER-2 polypeptide levels. A patient sample that has AIB1 or HER-2 polypeptide levels equal to or greater than such a control sample is said to have elevated AIB1 or HER-2 polypeptide levels.

As used herein, the expressions “cell”, “cell line” and “cell culture” are used interchangeably and all such designations include progeny. Thus, the words “transformant” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

The term “disease-free survival” as used herein is defined as a time between the first diagnosis and/or first surgery to treat a cancer patient and a first reoccurrence. For example, a disease-free survival is “low” if the cancer patient has a first reoccurrence within five years after tumor resection, and more specifically, if the cancer patient has less than about 55% disease-free survival over 5 years. For example, a high disease-free survival refers to at least about 55% disease-free survival over 5 years.

The term “endocrine therapy” as used herein is defined as a treatment of or pertaining to any of the ducts or endocrine glands characterized by secreting internally and into the bloodstream from the cells of the gland. The treatment may remove the gland, block hormone synthesis, or prevent the hormone from binding to its receptor.

The term “endocrine therapy-resistant patient” as used herein is defined as a patient receiving an endocrine therapy and lacks demonstration of a desired physiological effect, such as a therapeutic benefit, from the administration of an endocrine therapy.

The term “estrogen-receptor positive” as used herein refers to cancers that do have estrogen receptors while those breast cancers that do not possess estrogen receptors are “estrogen receptor-negative.”

The term “AIB1 levels” as used herein refers to either AIB1 polypeptide activity, functionality, or absolute numbers of polypeptide molecules. For example, elevated levels of AIB1 may be achieved when AIB1 polypeptide concentration is unchanged if AIB1 activity increases. Likewise, AIB1 levels may be elevated due to increased production and concentration of AIB1 polypeptide. The term “HER-2 levels” also refers to either HER-2 polypeptide activity or concentration. One with skill in the art realizes that AIB1 or HER-2 levels may increase as the result of increased transcription of corresponding mRNA. One with skill in the art also realizes that AIB1 or HER-2 levels may also change due to translational regulation or posttranlational modifications. Also, AIB1 or HER-2 levels may change due to changes in cellular localization.

The term “polypeptide” as used herein is used interchangeably with the term “protein” and is defined as a molecule which comprises more than one amino acid subunits. The polypeptide may be an entire protein or it may be a fragment of a protein, such as a peptide or an oligopeptide. The polypeptide may also comprise alterations to the amino acid subunits, such as methylation or acetylation.

The term “prognosis” as used herein are defined as a prediction of a probable course and/or outcome of a disease. For example, in the present invention AIB1 is a prognostic marker for resistance to endocrine therapy in a cancer patient.

The term “substantially similar to SEQ ID NO:1” as used herein is defined as a polypeptide having an amino acid sequence that is at least about 70% identical to or similar to SEQ ID NO:1, and the substantially similar polypeptide also exhibits the biological activity of the polypeptide of SEQ ID NO:1.

The term “substantially similar to SEQ ID NO:3” as used herein is defined as a polypeptide having an amino acid sequence that is at least about 70% identical to or similar to SEQ ID NO:3, and the substantially similar polypeptide also exhibits the biological activity of the polypeptide of SEQ ID NO:3.

The term “therapeutic benefit” as used herein refers to anything that promotes or enhances the well-being of the subject with respect to the medical treatment of his condition, which includes treatment of pre-cancer, cancer, and hyperproliferative diseases. A list of nonexhaustive examples of this includes extension of the subject's life by any period of time, decrease or delay in the neoplastic development of the disease, decrease in hyperproliferation, reduction in tumor growth, delay of metastases, reduction in cancer cell or tumor cell proliferation rate, and a decrease in pain to the subject that can be attributed to the subject's condition. In a specific embodiment, a therapeutic benefit refers to reversing de novo endocrine therapy-resistance or preventing the patient from acquiring an endocrine therapy-resistance.

The term “therapeutically effective amount” as used herein is defined as the amount of a molecule or a compound required to improve a symptom associated with a disease. For example, in the treatment of cancer such as breast cancer, a molecule or a compound which decreases, prevents, delays or arrests any symptom of the breast cancer is therapeutically effective. A therapeutically effective amount of a molecule or a compound is not required to cure a disease but will provide a treatment for a disease. A molecule or a compound is to be administered in a therapeutically effective amount if the amount administered is physiologically significant. A molecule or a compound is physiologically significant if its presence results in technical change in the physiology of a recipient organism.

The term “treatment” as used herein is defined as the management of a patient through medical or surgical means. The treatment improves or alleviates at least one symptom of a medical condition or disease and is not required to provide a cure.

The term “sample” as used herein indicates a patient sample containing at least one tumor cell. Tissue or cell samples can be removed from almost any part of the body. The most appropriate method for obtaining a sample depends on the type of cancer that is suspected or diagnosed. Biopsy methods include needle, endoscopic, and excisional. The treatment of the tumor sample after removal from the body depends on the type of detection method that will be employed for determining AIB1 or HER-2 levels.

Any of the methods described herein may be implemented using therapeutic compositions of the invention and vice versa. It is contemplated that any embodiment discussed with respect to an aspect of the invention may be implemented or employed in the context of other aspects of the invention.

II. The Present Invention

The present invention is directed to methods of identifying and treating breast cancer patients whose tumors may either have an endocrine therapy-resistantance phenotype (de novo resistance) or whose tumors will later develop endocrine-therapy resistance (acquired resistance), and in particular such a patient that is in need of an endocrine therapy. The invention is also appropriate for screening and treating breast cancer patients who are not endocrine therapy-resistant. The invention is based on Applicants discovery that an AIB1 polypeptide level in a tumor is correlated to disease-free survival and endocrine therapy-resistance of the patient.

A. Prognosis of Disease-Free Survival

In certain embodiments, the present invention is directed to a method of providing a prognosis of disease-free survival in a cancer patient not receiving an endocrine therapy, comprising the steps of obtaining a sample from the patient; and determining an AIB1 polypeptide level in the sample, wherein an elevated AIB1 polypeptide level as compared to a control sample indicates the prognosis of a high disease-free survival.

In further embodiments, the present invention comprises the step of measuring a HER-2 polypeptide level, wherein a low HER-2 polypeptide level and the high AIB1 polypeptide level as compared to the control indicates the prognosis of a high disease-free survival. In specific embodiments, the patient is not receiving a chemotherapy, radiotherapy or a hormonal therapy.

Another embodiment of the present invention is a method of providing a prognosis of disease-free survival in a cancer patient receiving an endocrine therapy comprising obtaining a sample from the patient; and determining an AIB1 polypeptide level in the sample, wherein an elevated AIB1 polypeptide level as compared to a control sample indicates the prognosis of a low disease-free survival. In further embodiments, a HER-2 polypeptide level is also determined in the sample, and an elevated AIB1 polypeptide level and an elevated HER-2 polypeptide level as compared to a control sample indicates the prognosis of a low disease-free survival.

In certain embodiments of the present invention, the cancer comprises an estrogen receptor-positive or a progesterone receptor-positive cancer. By this, the present invention encompasses patients having breast, ovarian, prostate, or endometrial cancer. It is contemplated that the patient has undergone a tumor resection, including a lumpectomy or a mastectomy in the case of breast cancer, although having had a tumor resection is not essential to the operability of the methods of the present invention.

In general, the sample comprises a fluid, a tissue or a cell, wherein the sample is obtained from the patient's body. In embodiments that involve a tumor, the sample that is analyzed by the methods described herein comprises a tumor cell, or a tissue or fluid therefrom.

In certain embodiments, the AIB1 polypeptide comprises an amino acid sequence substantially similar to SEQ ID NO:1 and includes a polypeptide having an amino acid sequence that is at least about 70% identical to or similar to SEQ ID NO:1. Further, the substantially similar polypeptide exhibits the biological activity of the polypeptide of SEQ ID NO:1 such as binding to an ER polypeptide to support cellular viability. One of ordinary skill in the art recognizes that in other embodiments of the present invention, a biologically active fragment of the AIB1 polypeptide is considered within the scope of the invention and of the term “substantially similar to SEQ ID NO:1”. In certain embodiments of the invention, a 130 kD alternative splice form of AIB is contemplated. See Reiter et al. One of ordinary skill in the art is aware of amino acid alterations that conserve the native chemical or biochemical characteristic of the native amino acid and this topic is discussed in more detail below.

In specific embodiments that involve measuring a HER-2 polypeptide level, the HER-2 polypeptide comprises an amino acid sequence substantially similar to SEQ ID NO:3. This includes a polypeptide having an amino acid sequence that is at least about 70% identical to or similar to SEQ ID NO:3, and the substantially similar polypeptide also exhibits the biological activity of the polypeptide of SEQ ID NO:3. HER-2 (also written HER-2/neu) is a growth factor found on the surface of cells that plays a key role in regulating cell growth. In a lifetime, a patient having the HER-2 gene that experiences an alteration of the HER-2 gene in the breast tissue which is at high-risk for developing breast cancer. In cases that lead to cancer, the alteration leads to the production of extra HER-2 receptors (i.e., elevated HER-2 polypeptide levels). The elevated HER-2 polypeptide levels cause cells to grow, divide, and multiply more rapidly than normal and may lead to breast cancer. Cancers that demonstrate an elevated HER-2 level tend to be aggressive and spread quickly to other regions of the patient's body. In other embodiments of the invention, HER-2 family polypeptides are contemplated. HER-2 is a member of a family of polypeptides receptors including EGFR, HER-2, HER-3 and HER-1 that interact to form an array of homo- and hetero-dimers. Thus, it is contemplated that increased signaling of any of the EGFR family polypeptides receptors in combination with increased AIB1 polypeptide levels may be used to make prognosis and treatment decisions in cancer patients who are receiving endocrine therapy, as well as for those for whom such treatment is an option. However, the effect of increasing AIB1 may not be limited to signaling withing the EGFR receptor family. It is contemplated that other cellular or growth factor signaling pathways or events may lead to increased levels of AIB1.

B. Predicting Endocrine Therapy-Resistance

Other embodiments of the present invention are directed to methods of identifying an endocrine therapy-resistant cancer patient comprising obtaining a tumor sample from the patient; and determining an AIB1 polypeptide level, wherein an elevated AIB1 polypeptide level as compared to a control sample indicates that the patient is endocrine therapy-resistant. In specific embodiments, the AIB1 polypeptide comprises an amino acid sequence substantially similar to SEQ ID NO:1.

In further embodiments, the method comprises the step of measuring a HER-2 polypeptide level, wherein an elevated HER-2 polypeptide level and the elevated AIB1 polypeptide level as compared to the control indicates that the patient is endocrine therapy-resistant. In specific embodiments that involve measuring a HER-2 polypeptide level, the HER-2 polypeptide comprises an amino acid sequence substantially similar to SEQ ID NO:3.

In other specific embodiments, the cancer comprises an estrogen receptor-positive or a progesterone receptor-positive cancer including, but not limited to, breast, ovarian, prostate, or endometrial cancer. In further specific embodiments, the patient has undergone a tumor resection, including a lumpectomy or a mastectomy in the case of breast cancer.

In specific embodiments, the sample is a fluid, a tissue or a cell. In another specific embodiment, the sample comprises a tumor fluid, tissue or cell.

C. Treatment Uses

Certain embodiments of the present invention provide a method of treating an endocrine therapy-resistant patient comprising administering therapeutically effective amounts of an endocrine therapy and of an antagonist of an AIB1 polypeptide. Other embodiments of the invention involve delaying or preventing the development of endocrine-therapy resistance.

In other embodiments the present invention provides a method of improving the effectiveness of an endocrine therapy in a endocrine-therapy resistant or non-resistant cancer patient comprising administering an therapeutically effective amount of an antagonist of an AIB1 polypeptide to the patient to provide a therapeutic benefit in the patient. The term “improving the effectiveness” refers to an increasing, augmenting, helping, promoting or enhancing the quality of a treatment of a disease or condition, or extending the amount of time that such a treatment may be effective. In the present invention, for example, if a cancer patient is identified as endocrine therapy-resistant, then the patient needs a means to improve, increase, promote or enhance the efficacy of an endocrine therapy in order to treat the disease. Thus, the cancer patient is administered a therapeutically effective amount of an antagonist of an AIB1 polypeptide. In doing such, the elevated AIB1 polypeptide levels that activates the estrogen receptors and lead to resistance to endocrine therapy are neutralized.

1. Cancer

The present invention is directed to treating a cancer patient in need of endocrine therapy, and in specific embodiments, the patient in need is endocrine therapy-resistant or predicited to be endocrine-therapy resistant. The treatment is directed to a cancer that is estrogen receptor-positive and/or progesterone receptor-positive because, in specific embodiments, these malignancies exhibit detectable expression of the ER coactivator AIB1. A skilled artisan is aware that estrogen and progesterone are steroid hormones. In a cancer of the present invention, the estrogen receptor-positive cancer requires a estrogen for growth and survival. In healthy individuals, progesterone is secreted by the corpus luteum of the ovary and by the placenta and acts to prepare the uterus for implantation of the fertilized ovum, to maintain pregnancy, and to promote development of secondary sexual characteristics. The presence of progesterone receptor also indicates a tissue that is being stimulated by estrogen because the progesterone receptor is induced by estrogen.

Non-limiting examples of endocrine therapies that are contemplated by the present invention include tamoxifen, raloxifene, or other SERMs (selective estrogen-receptor modulators). Tamoxifen has been the most commonly prescribed drug to treat breast cancer since its approval by the U.S. Food and Drug Administration (FDA) in the 1970s. Tamoxifen is an anti-estrogen and works by competing with the hormone estrogen to bind to estrogen receptors in breast cancer cells. Tamoxifen has been shown to reduce the risk of recurrence of an original cancer and the risk of developing new cancers by working against the effects of estrogen on breast cancer cells. A pharmaceutical composition comprising tamoxifen is generally administered as an oral composition such as a pill or capsule. Tamoxifen belongs to a class of agents known as selective estrogen receptor modulators. These agents display estrogen antagonist activity on some genes and agonist activity on others.

Raloxifene is another adjuvant employed in endocrine cancers and is an osteoporosis drug that has demonstrated activity in preventing the development of endocrine cancer.

In other specific embodiments, the endocrine therapy comprises goserelin acetate, leuprolide acetate, exemestane, megestrol, toremifene, fulvestrant, a nonsteroidal or a steroidal aromatase inhibitor including, for example, anastrozole and letrozole. Fulvestrant has demonstrated an ability to destroy estrogen receptors in breast cancer cells, and anastrozole prevents the production of estrogen in the fat and tumor tissue. In other specific embodiments, the patient is also administered another cancer therapy such as chemotherapy, radiotherapy, immunotherapy or gene therapy. More specifically, drugs and therapeutic agents that are contemplated are those that are useful for the treatment of estrogen receptor-positive or progesterone receptor-positive cancers including, but are not limited to, capecitabine, carboplatin, decetaxel, doxorubicin, epirubicin, paclitaxel, trastuzumab.

i. Anastrozole

Anastrozole has been approved for the treatment of advanced breast cancer in patients who have not responded well to treatment with tamoxifen. Anastrozole is usually administered in as an oral pharmaceutical composition.

ii. Exemestane

The FDA approved exemestane in 1999 to treat advanced (metastatic) breast cancer in post-menopausal women. The molecule works by binding to an aromatase enzyme in the metabolic pathway to produce estrogen to block biosynthesis of estrogen in vivo. This lack of estrogen “starves” cancer cells and prevents, hinders, or decreases their growth. Exemestane is usually administered as a oral pharmaceutical composition.

iii. Letrozole

Letrozole has been used as an endocrine therapy to treat advanced (metastatic) breast cancer in tumors that have not responded well to tamoxifen. Letrozole reduces the total amount of estrogen in the body (circulating estrogen levels), thereby limiting the amount of estrogen that can affect breast cancer cells. In post-menopausal women, the body produces estrogen from androgens by the enzymatic reaction of an aromatase. Letrozole is an aromatase inhibitor that blocks the aromatase from converting androgen into estrogen.

iv. Megestrol

Megestrol is a synthetic form of progesterone and has been used to treat breast and endometrial cancers. Megestrol serves to counteract some of the negative effects of estrogen in estrogen receptor-positive cancers.

v. Toremifene

Toremifene is an anti-estrogen or SERM, selective estrogen-receptor modulator that binds to estrogen receptors on estrogen receptor-positive cancer cells, thereby preventing the cells from growing and dividing. Toremifen is generally administered as an oral pharmaceutical composition having a therapeutically effective amount of about 60-milligram per dose.

vi. Goserelin Acetate

Goserelin acetate and leuprolide acete function by blocking estrogen from the ovary, thereby starving breast cancer cells fo estrogen. The drug is typically administered systemically by subcutaneous injection.

2. Combination Treatments

In the methods of the present invention directed to treating a cancer patient, it may be desirable to combine these treatments with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other treatments and/or compositions are provided in a combined amount effective to kill or inhibit proliferation of the cell. In specific embodiments, this process involves contacting the cells with a therapeutic construct, such as an expression construct comprising a therapeutic molecule, or a therapeutic molecule such as a drug, and the agent(s) or multiple factor(s) at the same time. This is achieved by contacting the cell with a single composition or pharmacological formulation that includes both agents, or by contacting the cell with two distinct compositions or formulations, at the same time, wherein one composition includes the expression construct and the other includes the second agent(s). For example, a chemotherapeutic agent is administered to the endocrine therapy-resistant cancer patient before, during or after endocrine therapy treatment.

Various combinations may be employed, an endocrine therapy and/or an antagonist of an AIB1 polypeptide is “A” and the secondary agent, such as a radiotherapeutic, chemotherapeutic, or gene therapy is “B”, such as tamoxifen and antagonist of AIB1 or something which enhances endocrine therapy:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of the therapeutic antagonist and/or antagonist of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

i. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabine, navelbine, famesyl-protein tansferase inhibitors, transplatinum, 5-fluorouracil, vincristin, vinblastin and methotrexate, or any analog or derivative variant of the foregoing.

Chemotherapeutics that are known in the art to treat advanced breast cancer include, but are not limited to capecitabine, cyclophosphamide, docetaxel, doxorubicin, epirubicin, and paclitaxel.

ii. Capecitabine

Capecitabine converts to a substance called 5-fluorouracil in situ and, in some patients, helps decrease tumor size by killing cancer cells. The FDA has approved capecitabine for the treatment of advanced breast cancer in patients who have not responded well to chemotherapy that included paclitaxel and an anthracycline such as doxorubicin.

iii. Cyclophosphamide

Cyclophosphamide is commonly used to many cancers because the mechanism of action involves first disrupting cancer cells, then destroying them. Cyclophosphamide is generally administered in a pharmaceutical composition suitable for oral or intravenously delivery.

iv. Docetaxel

Docetaxel is a cancer drug to treat cancer patients that have not responded well to chemotherapy, with doxorubicin, or have experienced spreading of the cancer into other parts of the body after treatment with standard chemotherapy. Docetaxel inhibits the division of breast cancer cells by acting on the cell's internal skeleton and is usually admininstered intravenously.

i. Doxorubicin

Doxorubicin is a chemotherapy drug commonly used to treat breast cancer and other cancers and function by first disrupting, then destroying the growth of cancer cells. Doxorubicin is generally administered intravenously.

ii. Epirubicin

Epirubicin decreases the likelihood of recurrence and increases a patient's disease-free survival. Epirubicin has been approved by the FDA to treat early-stage breast cancer after surgery, including a lumpectomy or a mastectomy, in patients whose cancer has spread to the axillary lymph nodes. Epirubicin is usually administered intravenously, and/or in combination with cyclophosphamide and fluorouracil.

iii. Paclitaxel

Paclitaxel has been approved by the FDA to treat both early and advanced breast cancer. Paclitaxel is a mitotic inhibitor because it interferes with mitosis (cell division). Paclitaxel is usually administered intravenously.

In certain embodiments, the administering of an endocrine therapy and/or an antagonist of an AIB1 polypeptide is combined with a chemotherapy regime. For example, chemotherapy regimes known in the art for the treatment of breast cancer include CMF therapy, which comprises administering cyclophosphamide, methotrexate and fluorouracil; CAF therapy, which comprises administering cyclophosphamide, doxorubicin,, and fluorouracil; AC therapy, which comprises administering doxorubicin and cyclophosphamide; administering doxorubicin and cyclophosphamide with paciltaxel; and administering doxorubicin (Adriamycin), followed by CMF cyclophosphamide, epirubicin and fluorouracil. Other chemotherapy drugs known in the art for treating breast cancer include docetaxel, vinorelbine, gemcitabine, and capecitabine.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. In the instant case, AIB1 is the preferred marker but other tumor markers are suitable.

An example of an immunotherapy that is contemplated for a combination treatment of the present invention is trastuzumab. Trastuzumab (Herceptin) has been used and is FDA approved for the treatment of advanced breast cancer, wherein the cancer demonstrates elevated levels of a HER-2 polypeptide. Trastuzumab binds to the HER-2 protein receptor on the surface of cells. By binding to the cells, Trastuzumab has been shown to slow the growth and spread of tumors that have an overabundance of HER-2 protein receptors and is usually administered intravenously.

4. Genes

In yet another embodiment, the secondary treatment is a gene therapy in which a therapeutic polynucleotide is administered before, after, or at the same time as the endocrine therapy and/or antagonist to the AIB1 polypeptide. Delivery of a vector encoding a therapeutic polynucleotide encoding one of the following gene products will have a combined anti-hyperproliferative effect on target tissues. Alternatively, if the antagonist is a polypeptide-encoded by a polynucleotide, then a single vector encoding both genes may be used. A variety of proteins are encompassed within the invention, some of which are described below.

5. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation. In another embodiment, it is contempalted that the antisense nucleotides are siRNAs.

The proteins FMS, ErbA, ErbB and HER-2/neu are growth factor receptors. Mutations or overexpression of these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth. The largest class of oncogenes includes the signal transducing proteins (e.g., Src, Abl and Ras). The proteins Jun, Fos and Myc are proteins that directly exert their effects on nuclear functions as transcription factors.

6. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are well known in the art as important inhibitors of cellular proliferation.

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

7. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists. Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

8. Antagonists of AIB1

The present invention is directed to treating a cancer patient by administering a therapeutically effective amount of an antagonist of an AIB1 polypeptide. The antagonist of the present invention blocks, interferes with or modulates an AIB1 biological or immunological activity, thereby rendering it unable to produce action on an estrogen receptor. Further, it is contemplated that the antagonists of the present invention reverse de novo resistance or prevent the patient from acquiring an endocrine therapy-resistance. The antagonist may dowregulate transcription or translation. The antagonist may act to degrade AIB1 transcripts. The antagonist may include proteins, peptides, soluble receptors, nucleic acids, carbohydrates, lipids, sugars, small molecules, haptens, steroids, or other molecules which bind to an AIB1 polypeptide or a biological equivalent thereof. The antagonist may be a compound that does not bind to AIB1, but inhibits its phosphorylation. In specific embodiments, the antagonist interferes with translation of the AIB1 polypeptide. In other embodiments, the antagonist interferes with an interaction between the AIB1 polypeptide and an estrogen receptor (ER) polypeptide. In yet other embodiments, the antagonist interferes with phosphorylation of the AIB1 polypeptide, which is essential for activation of the AIB1 polypeptide. In specific embodiments, the antagonist inhibits the function of a polypeptide encoding a kinase that specifically phosphorylates the AIB1 polypeptide. A skilled artisan recognizes that a kinase is an enzyme that effects the transformation of a phosphate moiety to a substrate. It is contemplated that at least one kinase functioning to phosphorylate the AIB1 polypeptide is inhibited to neutralize the AIB1 polypeptide levels in a cancer cell.

An example presented herein provides candidate substance screening methods that are based upon whole cell assays, in vivo analysis or transformed or immortal cell lines in which a reporter gene is employed to confer on its recombinant hosts a readily detectable phenotype that emerges only under conditions where an AIB1 gene is expressed at reduced levels relative to expression levels observed without the candidate substance. As an example, reporter genes encode a polypeptide not otherwise produced by the host cell that is detectable by analysis, e.g., by chromogenic, fluorometric, radioisotopic or spectrophotometric analysis. In a specific embodiment, the AIB1 nucleic acid sequence (SEQ ID NO:2) which encodes the AIB1 amino acid sequence (SEQ ID NO:1) has been replaced with β-galactosidase, which serves as a marker sequence.

In certain embodiments, the present invention provides a method of screening for a compound (e.g., an antagonist of AIB1 polypeptide). One embodiment is directed to identifying compounds that improve the effectiveness of an endocrine therapy in a patient and comprises the steps of introducing to a cell a test agent, wherein the cell comprises a polynucleotide encoding a AIB1 polypeptide under control of a promoter operable in the cell, and measuring the AIB1 polypeptide level, wherein when the level is decreased following the introduction, the test agent is the compound that improves effectiveness of the endocrine therapy in the patient.

Another example of a screening assay of the present invention is presented herein. AIB1 expressing cells are grown in microtiter wells, followed by addition of serial molar proportions of a candidate to a series of wells, and determination of the signal level after an incubation period that is sufficient to demonstrate expression in controls incubated solely with the vehicle which was used to resuspend or dissolve the compound. The wells containing varying proportions of candidate are then evaluated for signal activation. Candidates that demonstrate dose related reduction of reporter gene transcription or expression are then selected for further evaluation as clinical therapeutic agents.

In a specific embodiment, the candidate improves the effectiveness of an endocrine therapy in an endocrine therapy-resistant patient. Thus, the present invention also provides a method of treating an endocrine therapy-resistant cancer patient comprising: (i) identifying an antagonist of an AIB1 polypeptide introducing to a cell a test agent, wherein the cell comprises a polynucleotide encoding a AIB1 polypeptide under control of a promoter operable in the cell, and measuring the AIB1 polypeptide level, wherein when the level is decreased following the introduction, the test agent is the antagonist of the AIB1 polypeptide; and (ii) administering to the patient a therapeutically effective amount of the antagonist.

In an alternative embodiment there is a method for reducing an AIB1 nucleic acid level and consequently reducing an AIB1 polypeptide level by transfecting cells with antisense sequences of a sequence of AIB1 such as SEQ ID NO:2. Delivery systems for transfection of nucleic acids into cells may utilize either viral or non-viral methods. A targeted system for non-viral forms of DNA or RNA requires four components: 1) the DNA or RNA of interest; 2) a moiety that recognizes and binds to a cell surface receptor or antigen; 3) a DNA binding moiety; and 4) a lytic moiety that enables the transport of the complex from the cell surface to the cytoplasm. Further, liposomes and cationic lipids can be used to deliver the therapeutic gene combinations to achieve the same effect. Potential viral vectors include expression vectors derived from viruses such as adenovirus, vaccinia virus, herpes virus, and bovine papilloma virus. In addition, episomal vectors may be employed. Other DNA vectors and transporter systems are known in the art.

One skilled in the art recognizes that expression vectors derived from retroviruses, adenovirus, herpes or vaccinia viruses, or from various bacterial plasmids, may be used for delivery of nucleotides sequences to a targeted organ, tissue or cell population. Methods which are well known to those skilled in the art can be used to construct recombinant vectors that express antisense nucleotides of the gene encoding an AIB1 polypeptide. In specific embodiments, the gene is turned off by transfecting a cell or tissue with expression vectors which express high levels of a desired gene-encoding fragment. Such constructs can flood cells with untranslatable sense or antisense sequences. Even in the absence of integration into the DNA, such vectors may continue to transcribe RNA molecules until all copies are disabled by endogenous nucleases. Transient expression may last for a month or more with a non-replicating vector and even longer if appropriate replication elements are a part of the vector system.

Furthermore, the skilled artisan recognizes that modifications of gene expression can be obtained by designing antisense molecules to the control regions of an AIB1 nucleic acid sequence, i.e. the promoters, enhancers, and introns. Oligonucleotides derived from the transcription initiation site, e.g. between −10 and +10 regions of the leader sequence, are preferred. The antisense molecules may also be designed to block translation of mRNA by preventing the transcript from binding to ribosomes. Similarly, inhibition can be achieved by using “triple helix” base-pairing methodology. Triple helix pairing compromises the ability of the double helix to open sufficiently for the binding of polymerases, transcription factors, or regulatory molecules.

In specific embodiments, the antagonist is a ribozyme. Ribozymes are enzymatic RNA molecules capable of catalyzing the specific cleavage of RNA. The mechanism of ribozyme action involves sequence-specific hybridization of the ribozyme molecule to complementary target RNA, followed by endonucleolytic cleavage. Within the scope of the invention are engineered hammerhead motif ribozyme molecules that specifically and efficiently catalyze the endonucleolytic cleavage of sequences encoding a AIB1 polypeptide. In a specific embodiment, the ribozyme is a hammerhead motif ribozyme molecule against the 3′ untranslated region of the AIB1 gene. In another specific embodiment, the ribozyme leads to a decrease of ER-dependent activity and/or of tumor cell growth.

Antisense molecules and ribozymes of the invention may be prepared by any method known in the art for the synthesis of RNA molecules, including techniques for chemically synthesizing oligonucleotides. Alternatively, RNA molecules may be generated by in vitro and in vivo transcription of DNA sequences encoding an AIB1 polypeptide. Such DNA sequences may be incorporated into a wide variety of vectors with suitable RNA polymerase promoters such as T7 or SP6. Alternatively, antisense cDNA constructs that synthesize antisense RNA constitutively or inducibly can be introduced into cell lines, cells or tissues. Such a molecule may be an siRNA.

In a specific embodiment, the transfection of nucleic acid is facilitated by a transport protein, as described in Subramanian et al. (1999). Briefly, a peptide M9 is chemically bound to a cationic peptide as a carrier molecule. The cationic complex binds the negatively charged nucleic acid of interest, followed by binding of M9 to a nuclear transport protein, such as transportin.

In an alternative embodiment of the present invention an antagonist of an AIB1 polypeptide interferes with the interaction (binding) between the AIB1 polypeptide and an estrogen receptor polypeptide. A method to screen for anatgonist having this function, in one embodiment, involves an in vitro binding assay. For example, an AIB1 polypeptide is bound to a solid support; the AIB1 polypeptide is contacted with a second polypeptide, such as an estrogen receptor polypeptide, in the presence of a candidate substance; and the amount of the second polypeptide bound to the AIB1 polypeptide is determined, wherein a reduced amount as compared to a control assay under the same conditions indicates that the candidate substance is an antagonist of AIB1 and suitable for the present invention.

In other embodiments, screening methods involving a yeast two-hybrid expression system or an AIB1/ER co-precipitation assay using methods well-known to one of ordinary skill in the art are contemplated.

In an additional embodiment of the present invention the antagonist inhibits the biological activity of an AIB1 polypeptide comprising decreasing levels of a polypeptide that interacts with AIB1, such as a kinase or a kinase that activates the AIB1 polypeptide by effecting a phosphorylation thereof. In a specific embodiment, the antagonist decreases a nucleic acid sequence which has an AIB1 binding site in a regulatory region. In another embodiment, the antagonist interferes with the ability of AIB1 to activate the estrogen receptor.

One embodiment of the present invention is a method to administer antibodies to an AIB1 polypeptide, thereby preventing it from binding to an estrogen receptor. Such a method could be achieved by gene therapies known in the art and discussed herein or by administering an AIB1 antibody amino acid level or fragments thereof by methods standard in the art and also discussed herein.

One embodiment of the present invention is a method to administer compounds which affect an AIB1 polypeptide structure. Such compounds may include but are not limited to proteins, peptides, nucleic acids, carbohydrates, small molecules, haptens, steroids, sterols or other molecules which, upon binding to the AIB1 polypeptide, alter the AIB1 polypeptide structure, thereby rendering it ineffective in its activity.

One embodiment of the present invention is a method to administer a compound or compounds which affect an AIB1 polypeptide function. Such compounds may include but are not limited to proteins, peptides, nucleic acids, carbohydrates, small molecules, haptens, steroids, sterols or other molecules which upon binding inhibit or suppress function of an AIB1 polypeptide. An antagonist may interfere with AIB1 recruitment into complexes with the estrogen receptor on specific promoters of targeted genes.

D. Pharmaceutical Compositions

In certain aspects of the present invention, there are methods to treat a cancer patient comprising administering a therapeutically effective amount of an antagonist of AIB1 polypeptide and/or a therapeutically effective amount of an endocrine therapy. In such cases, the antagonist and/or the endocrine therapy may comprise a pharmaceutically acceptable carrier. By way of example and for ease of reading, the endocrine therapy in the following discussion is represented by a specific embodiment within the scope of the present invention, an adjuvant. This example is not intended in any way to limit the scope of the present invention with respect to pharmaceutical compositions or endocrine therapies of the present invention.

1. Pharmaceutically Acceptable Carriers

Aqueous compositions of the present invention comprise a therapeutically effective amount (used interchangably herein with the term “effective amount”) of an antagonist of an AIB1 polypeptide or an adjuvant dissolved and/or dispersed in a pharmaceutically acceptable carrier and/or aqueous medium.

The phrases “pharmaceutically and/or pharmacologically acceptable” refer to molecular entities and/or compositions that do not produce an adverse, allergic and/or other untoward reaction when administered to an animal, and/or a human, as appropriate.

As used herein, “pharmaceutically acceptable carrier” includes any and/or all solvents, dispersion media, coatings, antibacterial and/or antifungal agents, isotonic and/or absorption delaying agents and/or the like. The use of such media and/or agents for pharmaceutical active substances is well known in the art. Except insofar as any conventional media and/or agent is incompatible with the active ingredient, its use in the therapeutic compositions is contemplated. Supplementary active ingredients can also be incorporated into the compositions. For human administration, preparations should meet sterility, pyrogenicity, general safety and/or purity standards as required by FDA Office of Biologics standards.

If biological material is employed, it should be extensively dialyzed to remove undesired small molecular weight molecules and/or lyophilized for more ready formulation into a desired vehicle, where appropriate. The active compounds may generally be formulated for parenteral administration, e.g., formulated for injection via the intravenous, intramuscular, sub-cutaneous, intralesional, and/or even intraperitoneal routes. The preparation of an aqueous compositions that contain an effective amount of an antagonist of an AIB1 polypeptide and/or an adjuvant as an active component and/or ingredient will be known to those of skill in the art in light of the present disclosure. Typically, such compositions can be prepared as injectables, either as liquid solutions and/or suspensions; solid forms suitable for using to prepare solutions and/or suspensions upon the addition of a liquid prior to injection can also be prepared; and/or the preparations can also be emulsified.

The pharmaceutical forms suitable for injectable use include sterile aqueous solutions and/or dispersions; formulations including sesame oil, peanut oil and/or aqueous propylene glycol; and/or sterile powders for the extemporaneous preparation of sterile injectable solutions and/or dispersions. In all cases the form must be sterile and/or must be fluid to the extent that easy syringability exists. It must be stable under the conditions of manufacture and/or storage and/or must be preserved against the contaminating action of microorganisms, such as bacteria and/or fungi.

Solutions of the active compounds as free base and/or pharmacologically acceptable salts can be prepared in water suitably mixed with a surfactant, such as hydroxypropylcellulose. Dispersions can also be prepared in glycerol, liquid polyethylene glycols, and/or mixtures thereof and/or in oils. Under ordinary conditions of storage and/or use, these preparations contain a preservative to prevent the growth of microorganisms.

An antagonist of an AIB1 polypeptide and/or an adjuvant of the present invention can be formulated into a composition in a neutral and/or salt form. Pharmaceutically acceptable salts, include the acid addition salts (formed with the free amino groups of the protein) and/or which are formed with inorganic acids such as, for example, hydrochloric and/or phosphoric acids, and/or such organic acids as acetic, oxalic, tartaric, mandelic, and/or the like. Salts formed with the free carboxyl groups can also be derived from inorganic bases such as, for example, sodium, potassium, ammonium, calcium, and/or ferric hydroxides, and/or such organic bases as isopropylamine, trimethylamine, histidine, procaine and/or the like. In terms of using peptide therapeutics as active ingredients, the technology of U.S. Pat. Nos. 4,608,251; 4,601,903; 4,599,231; 4,599,230; 4,596,792; and/or 4,578,770, each incorporated herein by reference, may be used.

The carrier can also be a solvent and/or dispersion medium containing, for example, water, ethanol, polyol (for example, glycerol, propylene glycol, and/or liquid polyethylene glycol, and/or the like), suitable mixtures thereof, and/or vegetable oils. The proper fluidity can be maintained, for example, by the use of a coating, such as lecithin, by the maintenance of the required particle size in the case of dispersion and/or by the use of surfactants. The prevention of the action of microorganisms can be brought about by various antibacterial and/or antifungal agents, for example, parabens, chlorobutanol, phenol, sorbic acid, thimerosal, and/or the like. In many cases, it will be preferable to include isotonic agents, for example, sugars and/or sodium chloride. Prolonged absorption of the injectable compositions can be brought about by the use in the compositions of agents delaying absorption, for example, aluminum monostearate and/or gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the required other ingredients from those enumerated above. In the case of sterile powders for the preparation of sterile injectable solutions, the preferred methods of preparation are vacuum-drying and/or freeze-drying techniques which yield a powder of the active ingredient plus any additional desired ingredient from a previously sterile-filtered solution thereof. The preparation of more, and/or highly, concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and/or in such amount as is therapeutically effective. The formulations are easily administered in a variety of dosage forms, such as the type of injectable solutions described above, but drug release capsules and/or the like can also be employed.

For parenteral administration in an aqueous solution, for example, the solution should be suitably buffered if necessary and/or the liquid diluent first rendered isotonic with sufficient saline and/or glucose. These particular aqueous solutions are especially suitable for intravenous, intramuscular, subcutaneous and/or intraperitoneal administration. In this connection, sterile aqueous media which can be employed will be known to those of skill in the art in light of the present disclosure. For example, one dosage could be dissolved in 1 ml of isotonic NaCl solution and/or either added to 1000 ml of hypodermoclysis fluid and/or injected at the proposed site of infusion, (see for example, “Remington's Pharmaceutical Sciences” 15th Edition, pages 1035-1038 and/or 1570-1580). Some variation in dosage will necessarily occur depending on the condition of the subject being treated. The person responsible for administration will, in any event, determine the appropriate dose for the individual subject.

In addition to the compounds formulated for parenteral administration, such as intravenous and/or intramuscular injection, other pharmaceutically acceptable forms include, e.g., tablets and/or other solids for oral administration; liposomal formulations; time release capsules; and/or any other form currently used, including cremes.

One may also use nasal solutions and/or sprays, aerosols and/or inhalants in the present invention. Nasal solutions are usually aqueous solutions designed to be administered to the nasal passages in drops and/or sprays. Nasal solutions are prepared so that they are similar in many respects to nasal secretions, so that normal ciliary action is maintained. Thus, the aqueous nasal solutions usually are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation. Various commercial nasal preparations are known and/or include, for example, antibiotics and/or antihistamines and/or are used for asthma prophylaxis.

Additional formulations which are suitable for other modes of administration include vaginal suppositories and/or pessaries. A rectal pessary and/or suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions, suspensions, tablets, pills, capsules, sustained release formulations and/or powders. In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

The tablets, troches, pills, capsules and/or the like may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose as a sweetening agent methyl and/or propylparabens as preservatives, a dye and/or flavoring, such as cherry and/or orange flavor.

2. Lipid Formulations and/or Nanocapsules

In certain embodiments, the use of lipid formulations and/or nanocapsules is contemplated for the introduction of an AIB1 antagonist or an adjuvant into host cells. Liposomes interact with cells via four different mechanisms: Endocytosis by phagocytic cells of the reticuloendothelial system such as macrophages and/or neutrophils; adsorption to the cell surface, either by nonspecific weak hydrophobic and/or electrostatic forces, and/or by specific interactions with cell-surface components; fusion with the plasma cell membrane by insertion of the lipid bilayer of the liposome into the plasma membrane, with simultaneous release of liposomal contents into the cytoplasm; and/or by transfer of liposomal lipids to cellular and/or subcellular membranes, and/or vice versa, without any association of the liposome contents. Varying the liposome formulation can alter which mechanism is operative, although more than one may operate at the same time.

Nanocapsules can generally entrap compounds in a stable and/or reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and/or such particles may be easily made.

In a specific embodiment of the invention, the AIB1 antagonist may be associated with a lipid. The antagonist of an AIB1 polypeptide associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. The lipid or lipid/antagonist of an AIB1 polypeptide associated compositions of the present invention are not limited to any particular structure in solution. For example, they may be present in a bilayer structure, as micelles, or with a “collapsed” structure. They may also simply be interspersed in a solution, possibly forming aggregates which are not uniform in either size or shape.

Lipids are fatty substances which may be naturally occurring or synthetic lipids. For example, lipids include the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes.

Phospholipids may be used for preparing the liposomes according to the present invention and may carry a net positive, negative, or neutral charge. Diacetyl phosphate can be employed to confer a negative charge on the liposomes, and stearylamine can be used to confer a positive charge on the liposomes. The liposomes can be made of one or more phospholipids. Phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition, because of the instability and leakiness of the resulting liposomes.

A neutrally charged lipid can comprise a lipid with no charge, a substantially uncharged lipid, or a lipid mixture with equal number of positive and negative charges. Suitable phospholipids include phosphatidyl cholines and others that are well known to those of skill in the art.

Lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Preferably, chloroform is used as the only solvent since it is more readily evaporated than methanol.

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

Phospholipids can form a variety of structures other than liposomes when dispersed in water, depending on the molar ratio of lipid to water. At low ratios the liposome is the preferred structure. The physical characteristics of liposomes depend on pH, ionic strength and/or the presence of divalent cations. Liposomes can show low permeability to ionic and/or polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and/or results in an increase in permeability to ions, sugars and/or drugs.

Liposomes used according to the present invention can be made by different methods. The size of the liposomes varies depending on the method of synthesis. A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

Liposomes within the scope of the present invention can be prepared in accordance with known laboratory techniques. In one preferred embodiment, liposomes are prepared by mixing liposomal lipids, in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In the alternative, liposomes can be prepared in accordance with other known laboratory procedures: the method of Bangham et al. (1965), the contents of which are incorporated herein by reference; the method of Gregoriadis, as described in DRUG CARRIERS IN BIOLOGY AND MEDICINE, G. Gregoriadis ed. (1979) pp. 287-341, the contents of which are incorporated herein by reference; the method of Deamer and Uster (1983), the contents of which are incorporated by reference; and the reverse-phase evaporation method as described by Szoka and Papahadjopoulos (1978). The aforementioned methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated nucleic acid is removed by centrifugation at 29,000×g and the liposomal pellets washed. The washed liposomes are resuspended at an appropriate total phospholipid concentration, e.g., about 50-200 mM. The amount of nucleic acid encapsulated can be determined in accordance with standard methods. After determination of the amount of nucleic acid encapsulated in the liposome preparation, the liposomes may be diluted to appropriate concentrations and stored at 4° C. until use.

A pharmaceutical composition comprising the liposomes will usually include a sterile, pharmaceutically acceptable carrier or diluent, such as water or saline solution.

3. Dosage

The compounds (active ingredients) of this invention can be formulated and administered to treat a cancer patient, in particular an endocrine therapy-resistant cancer patient, by any means that produces contact of the active ingredient with the agent's site of action in the body of a vertebrate. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

The dosage administered will be a therapeutically effective amount of active ingredient and will, of course, vary depending upon known factors such as the pharmacodynamic characteristics of the particular active ingredient and its mode and route of administration; age, sex, health and weight of the recipient; nature and extent of symptoms; kind of concurrent treatment, frequency of treatment and the effect desired. The active ingredient(s) of the present invention include an AIB1 antagonist and/or an endocrine therapy, such as an adjuvant.

The active ingredient can be administered orally in solid dosage forms such as capsules, tablets and powders, or in liquid dosage forms such as elixirs, syrups, emulsions and suspensions. The active ingredient can also be formulated for administration parenterally by injection, rapid infusion, nasopharyngeal absorption or dermoabsorption. The agent may be administered intramuscularly, intravenously, subcutaneously, transdermally or as a suppository. In administering a compound, the compound may be given systematically. For compounds which require avoidance of systemic effects, a preferred embodiment is intrathecal administration. In a preferred embodiment, of the invention the compound is administered interarticularly for the treatment of arthritis.

Gelatin capsules contain the active ingredient and powdered carriers such as lactose, sucrose, mannitol, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

In general, water, a suitable oil, saline, aqueous dextrose (glucose), and related sugar solutions and glycols such as propylene glycol or polyethylene glycols are suitable carriers for parenteral solutions. Solutions for parenteral administration contain preferably a water soluble salt of the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

Additionally, standard pharmaceutical methods can be employed to control the duration of action. These are well known in the art and include control release preparations and can include appropriate macromolecules, for example polymers, polyesters, polyamino acids, polyvinyl, pyrrolidone, ethylenevinylacetate, methyl cellulose, carboxymethyl cellulose or protamine sulfate. The concentration of macromolecules as well as the methods of incorporation can be adjusted in order to control release. Additionally, the AIB1 antagonist and/or the adjuvant of the present invention can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Useful pharmaceutical dosage forms for administration of the compounds of this invention can be illustrated as follows. Pharmacological ranges for the active ingredients can be determined by the skilled artisan using methods well known in the art. Example ranges for the antagonist of an AIB1 polypeptide and/or the adjuvant may comprise about 0.0001 to 1.0 milligrams, and/or about 0.001 to 0.1 milligrams, and/or about 0.1 to 1.0 and/or even about 10 milligrams per dose and/or so. Multiple doses may be administered. Alternatively, a single dose is administered hourly, daily, weekly or monthly of a combination thereof. Alternatively, multiple doses are administered hourly, daily, weekly or monthly or a combination thereof. Example formulations are provided below, and are not intended to be limiting or exemplary formulations of the present invention:

Capsules: Capsules are prepared by filling standard two-piece hard gelatin capsulates each with powdered active ingredient, 175 milligrams of lactose, 24 milligrams of talc and 6 milligrams magnesium stearate.

Tablets: Tablets are prepared by conventional procedures so that the dosage unit contains the indicated amount of active ingredient, 0.2 milligrams of colloidal silicon dioxide, 5 milligrams of magnesium stearate, 275 milligrams of microcrystalline cellulose, 11 milligrams of cornstarch and 98.8 milligrams of lactose. Appropriate coatings may be applied to increase palatability or to delay absorption.

Injectable: A parenteral composition suitable for administration by injection is prepared by stirring 1.5% by weight of active ingredients in 10% by volume propylene glycol and water. The solution is made isotonic with sodium chloride and sterilized.

Suspension: An aqueous suspension is prepared for oral administration so that each 5 milliliters contains the indicated amount of finely divided active ingredient, 200 milligrams of sodium carboxymethyl cellulose, 5 milligrams of sodium benzoate, 1.0 grams of sorbitol solution U.S.P. and 0.025 milliliters of vanillin.

Accordingly, the pharmaceutical composition of the present invention may be delivered via various routes and to various sites in an animal body to achieve a particular effect. One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The composition of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and animal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

In a specific embodiment, a drug may be transported to a target by utilizing carbonic anhydrase inhibitor (CAI) which contains a polar group such as a carboxyl group, as described in Kehayova et al., 1999. The carboxyl group renders the composition dissolvable in water, however, upon exposure to light the bond linking the CAI to the carboxyl mask breaks, allowing the remaining portion to be soluble in a hydrophobic environment.

E. Methods to Screen

1. Screening for Modulators of Protein Function

The present invention comprises methods for identifying modulators of the function of an AIB1 polypeptide. These assays may comprise random screening of large libraries of candidate substances; alternatively, the assays may be used to focus on particular classes of compounds selected with an eye towards structural attributes that are believed to make them more likely to modulate the function of an AIB1 polypeptide.

By function, it is meant that one may assay for an substance or molecule that interferes with translation of the AIB1 polypeptide, that interferes with an interaction between the AIB1 polypeptide and an estrogen receptor polypeptide, that interferes with the activation of ER by AIB1, or that interferes with phosphorylation of the AIB1 polypeptide or other posttranlational modifications, including, for example, inhibiting the function of a polypeptide encoding a kinase that specifically phosphorylates the AIB1 polypeptide.

To identify an AIB1 polypeptide modulator, one generally will determine the function of the AIB1 polypeptide in the presence and absence of the candidate substance, a modulator defined as any substance that alters function. For example, a method generally comprises: providing a candidate modulator; admixing the candidate modulator with an isolated compound or cell, or a suitable experimental animal; measuring one or more characteristics of the compound, cell or animal; and comparing the characteristic measured with the characteristic of the compound, cell or animal in the absence of said candidate modulator, wherein a difference between the measured characteristics indicates that said candidate modulator is, indeed, a modulator of the compound, cell or animal. Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

2. Modulators

As used herein the term “candidate substance” refers to any molecule that may potentially inhibit an AIB1 polypeptide activity. The candidate substance may be a protein or fragment thereof, a small molecule, or even a nucleic acid molecule. It may prove to be the case that the most useful pharmacological compounds will be compounds that are structurally related to an estrogen receptor or derivative thereof. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory effect upstream, downstream or directly on an AIB1 polypeptide. Regardless of the type of antagonist identified by the present screening methods, the effect of the inhibition by such a compound results in reduced AIB1 polypeptide activity as compared to that observed in the absence of the added candidate substance.

3. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

4. In Cyto Assays

The present invention also contemplates the screening of compounds for their ability to modulate AIB1 polypeptide levels or activity in cells. Various cell lines can be utilized for such screening assays, including cells specifically engineered for this purpose. Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

5. In Vivo Assays

In vivo assays involve the use of various animal models, including, but not limited to, athymic mice, or transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

Thus, the present invention provides methods of screening for a candidate substance that functions as an antagonist of an AIB1 polypeptide. In these embodiments, the present invention is directed to a method for determining the ability of a candidate substance to function as an antagonist of an AIB1 polypeptide, generally including the steps of: administering a candidate substance to the animal; and determining the ability of the candidate substance to reduce one or more functional characteristics of an AIB1 polypeptide.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

6. Selectable and Screenable Markers

In certain embodiments of the invention, the cells contain nucleic acid constructs of the present invention, a cell may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

7. Host Cells

In certain embodiments of the present invention, in particularly those directed to screening for an antagonist of AIB1, a host cell is involved. As used herein, the terms “cell,” “cell line,” and “cell culture” also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials, which is readily accessible on the world wide web. An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

F. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce an elevated level of protein expression of a heterologous nucleic acid segment, such as described in U.S. Pat. Nos. 5,871,986 and 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®. Other examples of a bacterial expression systems are AFFINITY® T7 RNA polymerase-based pCAL vectors express cloned proteins as fusions with the calmodulin-binding peptide (CBP) tag and CLONTECH®'s HAT Protein Expression System.

Other examples of expression systems include STRATAGENE®'s COMPLETE CONTROL™ Inducible Mammalian Expression System, which involves a synthetic ecdysone-inducible receptor. Also from STRATAGENE® is the pET E. COLI EXPRESSION SYSTEM is a widely used in vivo bacterial expression system due to the strong selectivity of the bacteriophage T7 RNA polymerase, the high level of activity of the polymerase and the high efficiency of translation. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

Other assays may be used to identify responsive elements in a promoter region or gene. Such assays will be known to those of skill in the art (see for example, Sambrook et al., 1989; Zhang et al, 1997; Shan et al., 1997; Dai and Burnstein, 1996; Cleutjens et al., 1997; Ng et al., 1994; Shida et al., 1993), and include DNase I footprinting studies, Elecromobility Shift Assay patterns (EMSA), the binding pattern of purified transcription factors, effects of specific transcription factor antibodies in inhibiting the binding of a transcription factor to a putative responsive element, Western analysis, nuclear run-on assays, and DNA methylation interference analysis.

Gene expression may be determined by measuring the production of RNA, protein or both. The gene product (RNA or protein) may be isolated and/or detected by methods well known in the art. Following detection, one may compare the results seen in a given cell line or individual with a statistically significant reference group of non-transformed control cells. Alternatively, one may compare production of RNA or protein products in cell lines transformed with the same gene operably linked to various mutants of a promoter sequence. In this way, it is possible to identify regulatory regions within a novel promoter sequence by their effect on the expression of an operably linked gene.

1. Non-protein Expressing Sequences

DNA may be introduced into a host cell for the purpose of expressing RNA transcripts that function to affect a phenotype. Two examples are antisense RNA and RNA with ribozyme activity. Both may serve possible functions in reducing or eliminating expression of native or introduced genes.

2. Antisense RNA

Genes may be constructed or isolated, which when transcribed, produce antisense RNA that is complementary to all or part(s) of a targeted messenger RNA(s). The antisense RNA reduces production of the polypeptide product of the messenger RNA. The polypeptide product may be any protein encoded by the host's genome. The aforementioned genes will be referred to as antisense genes. An antisense gene may thus be introduced into a host cell by transformation methods known to a skilled artisan (i.e., electroporation) to reduce expression of a selected protein of interest. Reduction of the enzyme activity may reduce or eliminate products of the reaction which include any enzymatically synthesized compound in the host such as fatty acids, amino acids, carbohydrates, nucleic acids and the like. Alternatively, the protein may be a storage protein, or a structural protein, the decreased expression of which may lead to changes in amino acid composition or morphological changes respectively. The possibilities cited above are provided only by way of example and do not represent the full range of applications.

3. Ribozymes

Genes also may be constructed or isolated which, when transcribed, produce RNA enzymes (ribozymes) that can act as endoribonucleases and catalyze the cleavage of RNA molecules with selected sequences. The cleavage of selected messenger RNAs can result in the reduced production of their encoded polypeptide products. These genes may be used to reduced levels of polypeptides including, but not limited to, the polypeptides cited above.

Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al., 1981). For example, U.S. Pat, No. 5,354,855 reports that certain ribozymes can act as endonucleases with a sequence specificity greater than that of known ribonucleases and approaching that of the DNA restriction enzymes.

Several different ribozyme motifs have been described with RNA cleavage activity (Symons, 1992). Examples include sequences from the Group I self splicing introns including Tobacco Ringspot Virus (Prody et al., 1986), Avocado Sunblotch Viroid (Palukaitis et al., 1979), and Lucerne Transient Streak Virus (Forster and Symons, 1987). Sequences from these and related viruses are referred to as hammerhead ribozyme based on a predicted folded secondary structure.

Other suitable ribozymes include sequences from RNase P with RNA cleavage activity (Yuan et al., 1992, Yuan and Altman, 1994, U.S. Pat. Nos. 5,168,053 and 5,624,824), hairpin ribozyme structures (Berzal-Herranz et al., 1992; Chowrira et al., 1993) and Hepatitis Delta virus based ribozymes (U.S. Pat. No. 5,625,047). The general design and optimization of ribozyme directed RNA cleavage activity has been discussed in detail (Haseloff and Gerlach, 1988, Symons, 1992, Chowrira et al., 1994; Thompson et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozyme, the cleavage site is a dinucleotide sequence on the target RNA is a uracil (U) followed by either an adenine, cytosine or uracil (A,C or U) (Perriman et al., 1992; Thompson et al., 1995). The frequency of this dinucleotide occurring in any given RNA is statistically 3 out of 16. Therefore, for a given target messenger RNA of 1000 bases, 187 dinucleotide cleavage sites are statistically possible.

Designing and testing ribozymes for efficient cleavage of a target RNA is a process well known to those skilled in the art. Examples of scientific methods for designing and testing ribozymes are described by Chowrira et al., (1994) and Lieber and Strauss (1995), each incorporated by reference. The identification of operative and preferred sequences for use in down regulating a given gene is simply a matter of preparing and testing a given sequence, and is a routinely practiced “screening” method known to those of skill in the art.

4. Nucleic Acids and Uses Thereof

Certain aspects of the present invention concern at least one AIB1 nucleic acid SEQ ID NO:2 or biologically active variants thereof. In certain aspects, the at least one AIB1 nucleic acid comprises a wild-type or mutant AIB1 nucleic acid. In particular aspects, the AIB1 nucleic acid encodes for at least one transcribed nucleic acid. In certain aspects, the AIB1 nucleic acid comprises at least one transcribed nucleic acid. In particular aspects, the AIB1 nucleic acid encodes at least one AIB1 protein, polypeptide or peptide, or biologically functional equivalent thereof. In other aspects, the AIB1 nucleic acid comprises at least one nucleic acid segment of SEQ ID NO:2, or at least one biologically functional equivalent thereof.

The present invention also concerns the isolation or generation of at least one recombinant construct or at least one recombinant host cell through the application of recombinant nucleic acid technology known to those of skill in the art or as described herein. The recombinant construct or host cell may comprise at least one AIB1 nucleic acid, and may express at least one AIB1 protein, peptide or peptide, or at least one biologically functional equivalent thereof.

As used herein “wild-type” refers to the naturally occurring sequence of a nucleic acid at a genetic locus in the genome of an organism, and sequences transcribed or translated from such a nucleic acid. Thus, the term “wild-type” also may refer to the amino acid sequence encoded by the nucleic acid. As a genetic locus may have more than one sequence or alleles in a population of individuals, the term “wild-type” encompasses all such naturally occurring alleles. As used herein the term “polymorphic” means that variation exists (i.e., two or more alleles exist) at a genetic locus in the individuals of a population. As used herein “mutant” refers to a change in the sequence of a nucleic acid or its encoded protein, polypeptide or peptide that is the result of the hand of man.

A nucleic acid may be made by any technique known to one of ordinary skill in the art. Non-limiting examples of synthetic nucleic acid, particularly a synthetic oligonucleotide, include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986, and U.S. Pat. No. 5,705,629, each incorporated herein by reference. A non-limiting example of enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of oligonucleotides described in U.S. Pat. No. 5,645,897, incorporated herein by reference. A non-limiting example of a biologically produced nucleic acid includes recombinant nucleic acid production in living cells, such as recombinant DNA vector production in bacteria (see for example, Sambrook et al. 1989, incorporated herein by reference).

A nucleic acid may be purified on polyacrylamide gels, cesium chloride centrifugation gradients, or by any other means known to one of ordinary skill in the art (see for example, Sambrook et al. 1989, incorporated herein by reference).

The term “nucleic acid” will generally refer to at least one molecule or strand of DNA, RNA or a derivative or mimic thereof, comprising at least one nucleobase, such as, for example, a naturally occurring purine or pyrimidine base found in DNA (e.g. adenine “A,” guanine “G,” thymine “T” and cytosine “C”) or RNA (e.g. A, G, uracil “U” and C). The term “nucleic acid” encompass the terms “oligonucleotide” and “polynucleotide.” The term “oligonucleotide” refers to at least one molecule of between about 3 and about 100 nucleobases in length. The term “polynucleotide” refers to at least one molecule of greater than about 100 nucleobases in length. These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss”, a double stranded nucleic acid by the prefix “ds”, and a triple stranded nucleic acid by the prefix “ts.”

Thus, the present invention also encompasses at least one nucleic acid that is complementary to an AIB1 nucleic acid. In particular embodiments the invention encompasses at least one nucleic acid or nucleic acid segment complementary to the sequence set forth in SEQ ID NO:2. Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” also refers to nucleic acid(s) that are substantially complementary, as may be assessed by the same nucleotide comparison set forth above. The term “substantially complementary” refers to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double stranded nucleic acid molecule during hybridization. In certain embodiments, the term “substantially complementary” refers to at least one nucleic acid that may hybridize to at least one nucleic acid strand or duplex in stringent conditions. In certain embodiments, a “partly complementary” nucleic acid comprises at least one sequence that may hybridize in low stringency conditions to at least one single or double stranded nucleic acid, or contains at least one sequence in which less than about 70% of the nucleobase sequence is capable of base-pairing with at least one single or double stranded nucleic acid molecule during hybridization.

5. Assays of Gene Expression

Assays may be employed within the scope of the instant invention for determination of the relative efficiency of gene expression. For example, assays may be used to determine the efficacy of deletion mutants of specific promoter regions in directing expression of operatively linked genes. Similarly, one could produce random or site-specific mutants of promoter regions and assay the efficacy of the mutants in the expression of an operatively linked gene. Alternatively, assays could be used to determine the function of a promoter region in enhancing gene expression when used in conjunction with various different regulatory elements, enhancers, and exogenous genes.

Gene expression may be determined by measuring the production of RNA, protein or both. The gene product (RNA or protein) may be isolated and/or detected by methods well known in the art. Following detection, one may compare the results seen in a given cell line or individual with a statistically significant reference group of non-transformed control cells. Alternatively, one may compare production of RNA or protein products in cell lines transformed with the same gene operatively linked to various mutants of a promoter sequence. In this way, it is possible to identify regulatory regions within a novel promoter sequence by their effect on the expression of an operatively linked gene.

In certain embodiments, it will be desirable to use genes whose expression is naturally linked to a given promoter or other regulatory element. For example, a prostate specific promoter may be operatively linked to a gene that is normally expressed in prostate tissues. Alternatively, marker genes may be used for assaying promoter activity. Using, for example, a selectable marker gene, one could quantitatively determine the resistance conferred upon a tissue culture cell line or animal cell by a construct comprising the selectable marker gene operatively linked to the promoter to be assayed. Alternatively, various tissue culture cell line or animal parts could be exposed to a selective agent and the relative resistance provided in these parts quantified, thereby providing an estimate of the tissue specific expression of the promoter.

Screenable markers constitute another efficient means for quantifying the expression of a given gene. Potentially any screenable marker could be expressed and the marker gene product quantified, thereby providing an estimate of the efficiency with which the promoter directs expression of the gene. Quantification can readily be carried out using either visual means, or, for example, a photon counting device. Screenable markers are discussed in more detail below.

A preferred screenable marker gene for use with the current invention is β-glucuronidase (GUS). Detection of GUS activity can be performed histochemically using 5-bromo-4-chloro-3-indolyl glucuronide (X-gluc) as the substrate for the GUS enzyme, yielding a blue precipitate inside of cells containing GUS activity. This assay has been described in detail (Jefferson, 1987). The blue coloration can then be visually scored, and estimates of expression efficiency thereby provided. GUS activity also can be determined by immunoblot analysis or a fluorometric GUS specific activity assay (Jefferson, 1987). Similarly, 5-bromo-4chloro-3-indolyl galactoside (X-gal) is often used as a selectable marker, which confers a blue color on those transformants that comprise β-galactosidase activity.

G. Proteins, Peptides and Polypeptides

1. Proteinaceous Compositions

In certain embodiments, the present invention concerns at least one proteinaceous molecule, such as an AIB1 polypeptide or a HER-2 polypeptide or an ER polypeptide or an antagonist of an AIB1 polypeptide comprising a polypeptide, protein or peptide.

As used herein, a “proteinaceous molecule,” “proteinaceous composition,” “proteinaceous compound,” “proteinaceous chain” or “proteinaceous material” generally refers, but is not limited to, a protein or polypeptide of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments the size of the at least one proteinaceous molecule may comprise, but is not limited to, a molecule having about 5 to about 2500 or greater amino molecule residues, and any range derivable therein. The invention includes those lengths of contiguous amino acids of any sequence discussed herein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid.

In certain embodiments the proteinaceous composition comprises at least one protein, polypeptide or peptide. In methods that involve an antagonist of an AIB1 polypeptide, the anatgonist may comprise a protein, and as such, a composition comprising the antagonist is a proteinacious composition of the present invention. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases. The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

In certain embodiments, the proteinaceous composition may comprise at least a part of an antibody, for example, an antibody against a molecule expressed on a cell's surface, or against an AIB1 polypeptide. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. The term “antibody” is also used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Harlow et al., 1988; incorporated herein by reference).

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

2. Functional Aspects

When the present application refers to the function or activity of an AIB1 polypeptide, it is meant that the molecule in question is an estrogen receptor coactivator. One of ordinary skill in the art would further understand that this includes, for example, the ability to specifically bind to an estrogen receptor (ER) polypeptide. Determination of which molecules possess this activity may be achieved using assays familiar to those of skill in the art.

When the present application refers to the function or activity of an ER polypeptide, it is meant that the molecule in question is an estrogen receptor. One of ordinary skill in the art would further understand that this includes, for example, the ability to specifically bind an estrogen molecule. Determination of which molecules possess this activity may be achieved using assays familiar to those of skill in the art.

3. Variants of Proteinaceous Compositions

Amino acid sequence variants of the proteins, polypeptides and peptides of the present invention can be substitutional, insertional or deletion variants. Deletion variants lack one or more residues of the native protein that are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term “biologically functional equivalent” is well understood in the art and is further defined in detail herein. Accordingly, sequences that have between about 70% and about 80%; or more preferably, between about 81% and about 90%; or even more preferably, between about 91% and about 99%; of amino acids that are identical or functionally equivalent to the amino acids of the AIB1 polypeptide or ER polypeptide provided the biological activity of the protein is maintained. (see Table 1, below for a list of functionally equivalent codons).

TABLE 1

Codon Table

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamme | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity, as discussed below.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure. See e.g., Johnson (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of an AIB1 polypeptides or an antagonist of the AIB1 polypeptide, but with altered characteristics.

4. Fusion Proteins

A specialized kind of insertional variant is the fusion protein. This molecule generally has all or a substantial portion of the native molecule, linked at the N- or C-terminus, to all or a portion of a second polypeptide. In the present invention, a fusion may comprise an AIB1 sequence and a marker sequence. In other examples, fusions employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

Following transduction with an expression construct or vector according to some embodiments of the present invention, primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshner, 1992).

One embodiment of the foregoing involves the use of gene transfer to immortalize cells for the production and/or presentation of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Another embodiment of the present invention uses cell lines, which are transfected with an expression construct or vector that expresses a therapeutic protein such as a tumor suppressor. Examples of mammalian host cell lines include Vero and HeLa cells, other B- and T-cell lines, such as CEM, 721.221, H9, Jurkat, Raji, etc., as well as cell lines of Chinese hamster ovary, WI38, BHK, COS-7, 293, HepG2, 3T3, RIN and MDCK cells. In addition, a host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt- cells, respectively. Also, anti-metabolite resistance can be used as the basis of selection: for dhfr, which confers resistance to; gpt, which confers resistance to mycophenolic acid; neo, which confers resistance to the aminoglycoside G418; and hygro, which confers resistance to hygromycin.

Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large-scale production of cells and cell products. However, suspension cultured cells have limitations, such as tumorigenic potential and lower protein production than adherent cells.

5. Determining a Polypeptide or Protein Level

In certain embodiments of the present invention, the AIB1 polypeptide and/or HER-2 polypeptide levels are detected, measured or determined. One of ordinary skill in the art is aware of qualitative and quantitative methods and techniques to detect, measure, or determined a polypeptide level.

One specific example of a method to measure an AIB1 polypeptide level or a HER-2 polypeptide level involves extracting a frozen tumor sample from a cancer patient and performing a Western blot analysis on the sample. In other specific embodiments, a sample obtained from the cancer patient is analyzed by in situ hybridization. In other specific embodiments, the protein or polypeptide levels are detected using antibodies against AIB1 or HER-2 proteins, polypeptides and peptides, generally of the monoclonal type, that are linked to at least one agent to form an antibody conjugate.

In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain. It is known in the art that the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins”.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody-directed imaging”.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al, 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fe region have also been disclosed in the literature (O'Shannessy et al., 1987). This approach has been reported to produce diagnostically and therapeutically promising antibodies which are currently in clinical evaluation.

6. Immunodetection Methods

In still further embodiments, the present invention concerns immunodetection methods for binding, purifying, removing, quantifying and/or otherwise generally detecting biological components such as AIB1 protein components. The AIB1 antibodies prepared in accordance with the present invention may be employed to detect wild-type and/or mutant AIB1 proteins, polypeptides and/or peptides. As described throughout the present application, the use of wild-type and/or mutant AIB1 specific antibodies is contemplated. Some immunodetection methods include enzyme linked immunosorbent assay (ELISA), radioimmunoassay (RIA), immunoradiometric assay, fluoroimmunoassay, immunohistoligical assay, chemiluminescent assay, bioluminescent assay, and Western blot to mention a few. The steps of various useful immunodetection methods have been described in the scientific literature, such as, e.g., Doolittle M H and Ben-Zeev 0, 1999; Gulbis B and Galand P, 1993; De Jager R et al., 1993; and Nakamura et a., 1987, each incorporated herein by reference.

In general, the immunobinding methods include obtaining a sample suspected of containing AIB1 protein, polypeptide and/or peptide, and contacting the sample with a first anti-AIB1 antibody in accordance with the present invention, as the case may be, under conditions effective to allow the formation of immunocomplexes.

These methods include methods for purifying wild-type and/or mutant AIB1 proteins, polypeptides and/or peptides as may be employed in purifying wild-type and/or mutant AIB1 proteins, polypeptides and/or peptides from patients' samples and/or for purifying recombinantly expressed wild-type or mutant AIB1 proteins, polypeptides and/or peptides. In these instances, the antibody removes the antigenic wild-type and/or mutant AIB1 protein, polypeptide and/or peptide component from a sample. The antibody will preferably be linked to a solid support, such as in the form of a column matrix, and the sample suspected of containing the wild-type or mutant AIB1 protein antigenic component will be applied to the immobilized antibody. The unwanted components will be washed from the column, leaving the antigen immunocomplexed to the immobilized antibody, which wild-type or mutant AIB1 protein antigen is then collected by removing the wild-type or mutant AIB1 protein and/or peptide from the column.

In other embodiments, the methods of screening for an antagonist of AIB1 that interferes with the binding between the AIB1 and an ER polypeptide comprise fixing a recombinantly expressed wild-type or mutant AIB1 proteins, polypeptides and/or peptides to a solid support, such as in the form of a column matrix, in the presence of a candidate substance, and the sample containing the wild-type ER protein component will be applied to the immobilized AIB1 polypeptide. The unwanted components will be washed from the column, leaving the ER protein component uncomplexed to the immobilized AIB1 polypeptide if the candidate substance is a suitable AIB1 antagonist.

The immunobinding methods also include methods for detecting and quantifying the amount of a wild-type or mutant AIB1 protein reactive component in a sample and the detection and quantification of any immune complexes formed during the binding process. Here, one would obtain a sample suspected of containing a wild-type or mutant AIB1 protein and/or peptide, and contact the sample with an antibody against wild-type or mutant AIB1, and then detect and quantify the amount of immune complexes formed under the specific conditions.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any AIB1 protein antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The AIB1 antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. Instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

The immunodetection methods of the present invention have evident utility in the prognosis of a cancer patient, predicting endocrine therapy-resistance in a cancer patient and/or screening for an antagonist to the AIB1 polypeptide. Here, a biological and/or clinical sample suspected of containing a wild-type or mutant AIB1 protein, polypeptide, peptide and/or mutant is used. However, these embodiments also have applications to non-clinical samples, such as in the titering of antigen or antibody samples, for example in the selection of hybridomas.

In the clinical prognosis and/or identification of patients with various forms of cancer, such as estrogen receptor-positive or progesterone receptor-positive cancers, that need endocrine therapy, the detection of AIB1 mutant, and/or an alteration in the levels of AIB1 polypeptide levels, in comparison to the levels in a corresponding biological sample from a normal subject is indicative of a patient with cancer, such as estrogen receptor-positive or progesterone receptor-positive cancers. However, as is known to those of skill in the art, such a clinical prognosis would not necessarily be made on the basis of this method in isolation. Those of skill in the art are very familiar with differentiating between significant differences in types and/or amounts of other biomarkers, which are useful to establish background levels. Indeed, background expression levels are often used to form a “cut-off” above which increased detection will be scored as significant and/or positive.

7. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used. Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

8. Immunohistochemistry

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

### EXAMPLES

The following examples are included to demonstrate preferred embodiments of the invention. It should be appreciated by those skilled in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Characteristics of Patients and Tumors

For initial correlations with AIB1, all 316 specimens were used. Table 2 shows the characteristics of the entire study population and the treatment subsets. The majority of patients in this selected study population were above age 50. By definition, all had positive axillary lymph nodes, with a little more than half having only 1-3 positive nodes. Most of the patients had small or intermediate size tumors and tumors with intermediate or high S-phase fraction. 89% were ER-positive (reflecting the selection criteria), while 62% of tumors expressed PR. 31% had intermediate or high levels of HER-2. The characteristics of the treatment subsets were very similar to the total group. The patient population exhibited a slightly greater proportion of ER and/or PR positive tumors in the endocrine therapy subset.

A few patients treated with adjuvant chemotherapy were included in the all patients category and were used for initial correlations, but they are not included in the untreated or tamoxifen therapy groups.

TABLE 2
Patient and Tumor Characteristics

| | Untreated Patients | | Tamoxifen Therapy | | All Patients | |
|---|---|---|---|---|---|---|
| Variable | N | % | N | % | N | % |
| Age | | | | | | |
| ≧50 yr | 95 | 80% | 168 | 90% | 269 | 85% |
| <50 yr | 24 | 20% | 19 | 10% | 47 | 15% |
| Nodes | | | | | | |
| 1-3 | 64 | 54% | 104 | 56% | 172 | 55% |
| >3 | 54 | 46% | 83 | 44% | 143 | 45% |
| Tumor size | | | | | | |
| ≦2 cm | 23 | 20% | 51 | 27% | 78 | 25% |
| 2-5 cm | 69 | 58% | 107 | 57% | 180 | 57% |
| >5 cm | 26 | 22% | 29 | 16% | 57 | 18% |
| S-phase fraction | | | | | | |
| Low (<6%) | 20 | 17% | 49 | 27% | 72 | 23% |
| Intermediate (6-10%) | 41 | 35% | 59 | 32% | 101 | 33% |
| High (>10%) | 57 | 48% | 75 | 41% | 137 | 44% |
| ER | | | | | | |
| Positive | 96 | 81% | 177 | 95% | 281 | 89% |
| Negative | 23 | 19% | 10 | 5% | 35 | 11% |
| PgR | | | | | | |
| Positive | 58 | 53% | 122 | 66% | 187 | 62% |
| Negative | 52 | 47% | 62 | 34% | 116 | 38% |
| P53 IHC Score | | | | | | |
| 0-4 | 88 | 76% | 160 | 86% | 256 | 82% |
| 5-8 | 28 | 24% | 26 | 14% | 55 | 18% |
| HER-2 units | | | | | | |
| 0-1 | 84 | 71% | 127 | 68% | 218 | 69% |
| 2-4 | 35 | 29% | 60 | 32% | 97 | 31% |
| Bcl 2 IHC Score | | | | | | |
| 0-4 | 50 | 44% | 72 | 40% | 125 | 41% |
| 5-8 | 63 | 56% | 108 | 60% | 177 | 59% |
| AIB1 | | | | | | |
| 1^{st} quartile | 30 | ≦0.79 | 48 | ≦0.76 | 84 | ≦.78 |
| 2^{nd} quartile | 30 | >0.79-1.17 | 46 | >0.76-1.11 | 85 | >.78-1.15 |
| 3^{rd} quartile | 30 | >1.17-1.67 | 47 | >1.11-1.55 | 79 | >1.15-1.61 |
| 4^{th} quartile | 29 | >1.67 | 46 | >1.55 | 68 | >1.61 |

### Example 2

### AIB1 Correlations with Molecular Markers and Clinical Variables

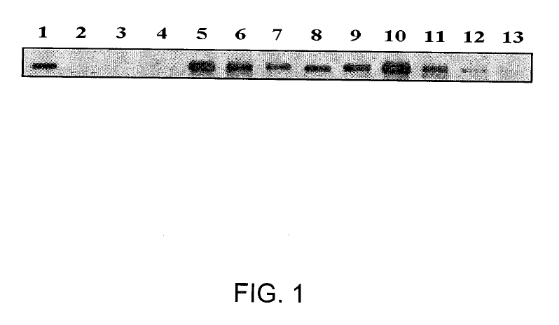

Expression of AIB1 in different tumors varied widely (FIG. 1). Normalized band intensity ranged from 0.26 to a maximum of 5.7 (more than a 20 fold difference). The mean score was 1.3 and the median 1.15.

Examination of the correlations of various molecular markers and clinical variables in the tumor specimens indicated ER expression positively correlated with age, PR, and bcl-2, and inversely correlated with p53, S-phase fraction, and tumor size. AIB1 protein expression, on the other hand, demonstrated a weak inverse correlation with PR and no correlation with ER (Table 3). Statistically significant direct correlations were observed for AIB1 with S-phase fraction and HER-2 expression. The direct correlations between AIB1 and several factors associated with a typically more aggressive phenotype indicated that AIB1 expression correlates with relatively poor prognosis.

TABLE 3
Correlations of AIB1 Expression with Molecular Markers
and Clinical Variables

| | AIB1 | |
|---|---|---|
| | Correlation | |
| Variable | Coefficient* | P-Value |
| ER | −0.07 | .20 |
| PR | −0.11 | .05 |
| p53 | 0.09 | .10 |
| HER-2 | 0.22 | <.0001 |
| Age | 0.008 | .88 |
| Tumor size | 0.09 | .09 |
| S-phase | 0.21 | .003 |
| bcl-2 | −0.08 | .15 |
| Nodes | 0.07 | .22 |

*Spearman Correlation Coefficient; all variables are continuous.

### Example 3

### AIB1 and Prognosis: Clinical Outcome in Patients Treated with Local Therapy Only

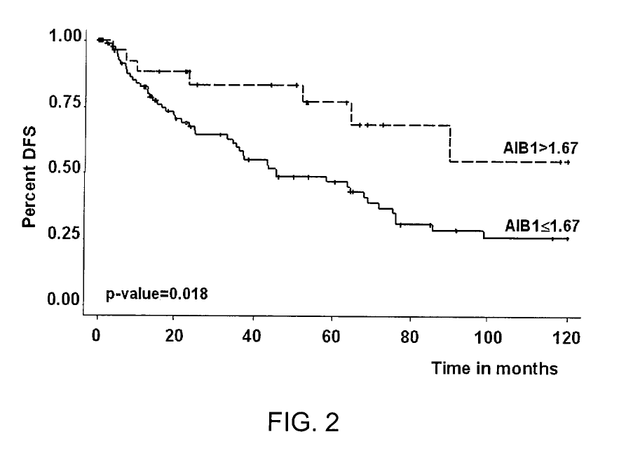

A subset of 119 patients receiving only local treatment was analyzed for AIB1 expression levels. The exclusion of patients receiving adjuvant therapy allowed discrimination between the prognostic (natural progression) and predictive (response to drug) contributions of AIB1 expression. In spite of previous expectation that high AIB1 expression might correlate with relatively poor outcome, elevated levels were associated with a better prognosis—the five-year DFS for patients in the highest quartile of AIB1 expression was 77% compared to 47% for those in the other three quartiles (P=0.018) (FIG. 2). The highest quartile of AIB1 expression (AIB1>1.67) has significantly better outcome than the lower three quartiles (AIB1≦1.67) (P=0.018). When AIB1 expression was considered as a continuous variable, and a trend for higher AIB1 expression to be associated with a better prognosis (P=0.07) was observed. AIB1 expression was not, however, significantly related to overall survival, an endpoint which is confounded by multiple treatments given after recurrence.

Other factors in this patient subset which had statistically significant correlations with favorable DFS included status of ER (P=0.0079) and PR (P=0.017). Unfavorable outcome was predicted by high HER-2 scores (P=0.024), high S-phase fraction (P=0.013), multiple positive lymph nodes (P<0.0001), and larger tumor size (P=0.004). Including these six factors and AIB1 in a Cox multivariate analysis of DFS in which the values for AIB1, ER, PR, HER-2, number of nodes, and tumor size were dichotomized, only nodes, ER, AIB1, and HER-2 remained statistically significant (Table 4)—more than 3 positive lymph nodes, negative ER status, low AIB1, and elevated expression of HER-2 were all associated with an increased risk of recurrence. In a similar analysis in which AIB1, ER, PR, and S-phase were described as continuous variables, nodes (P<0.0001), HER-2 status (P=0.022), and AIB1 (P=0.037) remained statistically significant, though ER was replaced by tumor size (P=0.026). AIB1 protein expression was still not related to overall survival, although nodal status, ER, HER-2, and tumor size were significant. Thus, in patients with no adjuvant therapy, elevated AIB1 expression was associated with a more indolent tumor and more favorable patient outcome.

TABLE 4
Multivariate Analyses* of Disease-Free Survival
No Adjuvant Therapy (108 patients, 47 recurrences)

| Variable | Hazard Ratio | 95% CI | P-Value |
|---|---|---|---|
| Nodes >3 | 4.04 | 2.16-7.58 | <0.0001 |
| ER negative | 2.84 | 1.36-5.96 | 0.0033 |
| AIB1 (top quartile) | 0.31 | 0.13-0.75 | 0.0055 |
| HER-2 (grade 2-4) | 1.90 | 1.02-3.51 | 0.0391 |

*Cox multivariate analyses, forward selection

### Example 4

### AIB1 in Predicting Tamoxifen Benefit: Tumors From Patients Treated With Adjuvant Tamoxifen

The effect of AIB1 on tamoxifen benefit was examined using 187 tumors from patients receiving tamoxifen adjuvant therapy. The mean and median values for AIB1 expression in these patients (mean=1.23; median=1.1) were very similar to those observed for the untreated patients. However, in contrast to the untreated patients, AIB1 was not a favorable factor for disease-free survival in this group. The highest AIB1 quartile tended to have a worse outcome, although the difference did not reach significance. However, statistical significance was attained when the 10 known ER-negative cases were excluded and AIB1 was considered as a continuous variable (hazard rate 1.37, P=0.049). Continuous AIB1 remained significantly adverse in a multivariate analysis, along with nodes >3, while ER, PR, HER-2, and S-phase fraction did not contribute significantly. As in the untreated group, AIB1 was not a predictor of overall survival in these adjuvant tamoxifen patients.

Thus, although elevated AIB1 predicted a more indolent course for patients receiving no adjuvant therapy, elevated levels predict worse DFS in patients receiving tamoxifen. A test for interaction between AIB1 and treatment on the treated and untreated groups combined was highly significant (P=0.0039), formally confirming that the effect of AIB1 is different in the tamoxifen-treated patients. These data indicated that tumors expressing abundant AIB1 are tamoxifen resistant.

### Example 5

### Interaction of AIB1 and HER-2

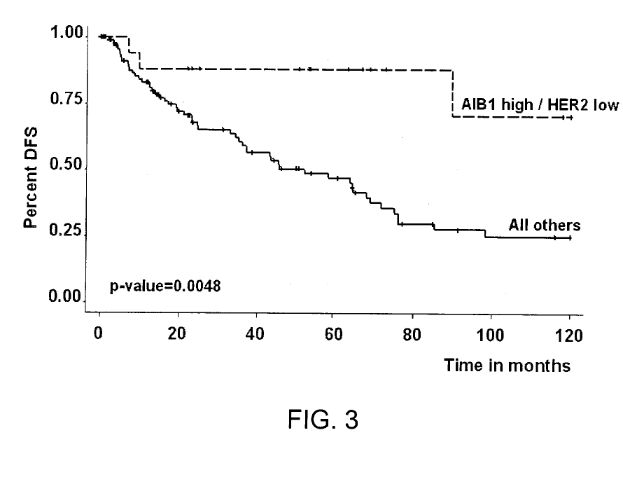

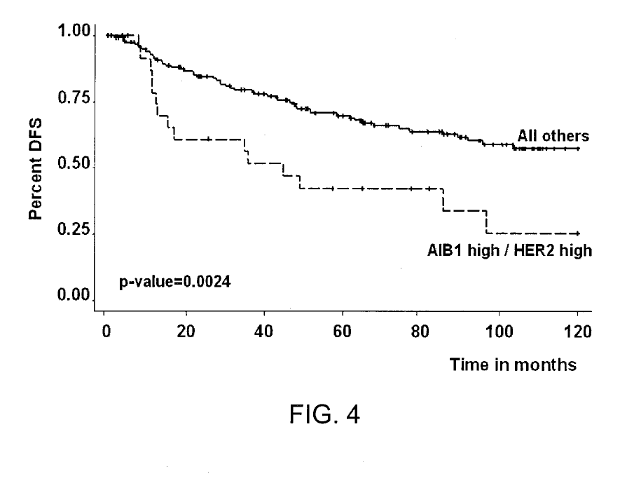

A consequence of HER-2 signaling is activation of AIB1. Considering first the interaction of HER-2 and AIB1 in patients receiving tamoxifen (Table 5A), the data indicate that HER-2 is low and AIB1 predicts better DFS. FIG. 3 shows that the patients in the elevated AIB1/low HER-2 subset had remarkably better DFS as compared to the other patients. The patients did not receive adjuvant therapy and those with elevated AIB1 (highest quartile) and low HER-2 (grades 0-1) have much better outcome than all other groups (P=0.0048). High HER-2 is indeed an adverse factor in these patients, but the data indicates that AIB1 does not further affect the patients' outcomes. Patients receiving adjuvant tamoxifen (Table 5B), having low HER-2 expression demonstrated that AIB1 levels indicated favorable DFS although the difference in this case was not significant. However, AIB1 was a distinctly adverse factor in patients having an elevated HER-2 level. A Kaplan-Meier DFS plot comparing the group with elevated expression of both proteins with the other three groups is shown in FIG. 4. As shown, those patients with elevated AIB1 (highest quartile) and high HER-2 (grades 2-4) had much worse outcome than all other groups (shown in FIGS. 3 and 4). The 5-year DFS of the group with elevated expression of both proteins (42%) was much worse than the other three groups combined (70%). The difference between these curves is highly significant (P=0.002). Interestingly, the group with elevated levels of HER-2, but low levels of AIB1, had an excellent outcome despite HER-2 overexpression (5-year DFS 77%). Thus, in the presence of tamoxifen, HER-2 is an indicator of poor outcome and, therefore, of tamoxifen resistance only if elevated levels of AIB1 are available to mediate the adverse effect.

TABLE 5
Five-year Disease-free Survival by AIB1 and HER-2 Status
with or without Tamoxifen Adjuvant Therapy

| | n | 5 yr DFS | 95% CI | P-value |
|---|---|---|---|---|
| A) No Adjuvant Therapy | | | | |
| (n = 119) | | | | |
| High HER-2 (grade 2-4) | | | | |
| High AIB1 (top quartile) | 10 | 36% | 0-88% | 0.40 |
| Low AIB1 (lower 3 quartiles) | 25 | 33% | 13-54% | |
| Low HER-2 (grade 0-1) | | | | |
| High AIB1 (top quartile) | 19 | 88% | 72-100% | 0.013 |
| Low AIB1 (lower 3 quartiles) | 65 | 53% | 39-67% | |
| B) Tamoxifen Adjuvant Therapy | | | | |
| (n = 187) | | | | |
| High HER-2 (grade 2-4) | | | | |
| High AIB1 (top quartile) | 25 | 42% | 22-63% | 0.003 |
| Low AIB1 (lower 3 quartiles) | 35 | 77% | 63-92% | |
| Low HER-2 (grade 0-1) | | | | |
| High AIB1 (top quartile) | 21 | 82% | 61-100% | 0.20 |
| Low AIB1 (lower 3 quartiles) | 106 | 64% | 54-74% | |

### Example 6

### AIB1 is a Biomarker for Hormonal Therapies

The prognostic significance of AIB1 was evaluated in patients randomly assigned to no systemic adjuvant therapy, and the predictive significance was evaluated by testing the interaction between AIB1 and treatment. A clinically useful and validated assay for AIB1 was developed based on the data collected from the patient population. It is important to determine whether levels of AIB1 and HER-2 are determined and queried for a correlation with response to endocrine therapies that work by varying mechanisms. ER coactivator levels and ligand-independent activation of ER induced by the HER-2 pathway are also investigated for therapies such as aromatase inhibitors designed to reduce the estrogen concentration (the natural ligand for ER). Treatment with SERMs, which are receptor ligands which have mixed agonist and antagonist properties on ER are also contemplated. Although ER and PR are clinically useful tumor markers, many patients with ER-positive tumors fail to benefit from hormonal therapies, and other biomarkers are needed to distinguish these patients. This study indicates that AIB1 is biomarker and therapeutic target for cancer patients in need of hormonal (endocrine) therapy.

### Example 7

### AIB1 and Disease-Free Survival

A comparison of protein expression levels of the ER coregulator AIB1 with tumor and clinical variables in patients with primary breast cancer indicated that AIB1 is a prognostic marker for disease-free survival in a cancer patient receiving adjuvant therapy and a predictor of resistance to endocrine therapy. The tumor specimens were not derived from patients randomized to no adjuvant treatment or to tamoxifen, and many of the analyses are exploratory in nature, however, these results indicate that ER coregulators are important in the pathophysiology of disease in humans.

Patients diagnosed with ER-positive primary breast cancer and treated by local therapies followed by adjuvant tamoxifen demonstrated elevated levels of the coactivator AIB1 that correlated significantly with poor DFS in both univariate and multivariate analyses. These data lend support to the hypothesis that the estrogen agonist activity of SERMs, such as tamoxifen, are enhanced by inducing higher levels of coactivators thereby rendering the drug less antiestrogenic. Multivariate analyses of these primarily ER-positive patients indicated that AIB1 was even more important than PR levels and HER-2 levels, which are molecular markers previously shown to predict tamoxifen benefit or resistance (Mass, R., 2000; Ciocca, D. R. and Elledge, R., 2000). These results are even more striking if viewed in the context that patients treated only by surgery and no tamoxifen, high AIB1 levels predicted a less aggressive clinical course and better patient outcome. As such, the test for interaction between AIB1 and tamoxifen treatment or not was highly significant.

### Example 8

### Endocrine Therapy Resistance and AIB1 and HER-2

There is ample biologic evidence to indicate a mechanism in which coactivators such as AIB1 modulate the estrogen agonist or antagonist properties of SERMs, such as tamoxifen, to reduce antitumor activity in patients. The present invention shows that AIB1 contributes to a reduced benefit from tamoxifen patients with ER-positive tumors which also express high levels of the HER-2 receptor. Signaling through the epidermal growth factor/HER-2 receptor family activates the ERK 1, 2 mitogen-activated protein kinase that has been shown, in turn, to phosphorylate not only ER, but also AIB1 (Font de Mora et al., 2000). No correlation between AIB1 and ER concentration was observed, but a positive correlation with HER-2 was observed at the protein level. The direct correlation between AIB1 and HER-2 protein levels, which has also been observed at the RNA level in other studies (Bouras et al., 2001), and the exceptionally worse outcome for tamoxifen-treated patients having tumors express high levels of both AIB1 and HER-2, are clinically important.

One embodiment of the present invention is directed to a method of predicting cancer treatment outcomes in tumors by detecting expression levels of HER-2 and AIB1 wherein high expression levels of both indicate a patient resistant to tamoxifen treatment. The inconsistent results in prior studies which measured HER-2 in tamoxifen-treated patients failed in part, because consideration of AIB1, which is necessary to enhance tamoxifen's agonist activity, was not made, and, further, because of the lack of assay standardization for the measurement of HER-2. As described herein, those tumors characterized by high HER-2 expression and relatively low AIB1, responded to tamoxifen adjuvant therapy.

One embodiment of the present invention is directed to a method of determining the levels of HER-2 expression relative to AIB1 necessary for the tamoxifen-resistant tumor phenotype. The highly amplified levels found in some breast cancers that are necessary for tumor response to HER-2 targeted therapies are determined to induce or not induce a more estrogen-agonist function of tamoxifen-bound ER. Furthermore, given the relatively high EGF receptor expression in normal breast ductal epithelium, AIB1 levels in normal and premalignant breast epithelium are tested for implications on the effectiveness of tamoxifen in cancer prevention.

### Example 9

### Patients and Tumor Specimens

Frozen tumor specimens from 316 patients, all of whom had positive axillary lymph nodes at the time of initial surgery, were selected for study. Such patients have higher recurrence rates after primary treatment, resulting in more events for statistical power. A subset of these patients (n=19) had received no adjuvant chemotherapy or tamoxifen after primary surgery, and another group of 187 patients had received tamoxifen after primary surgery. The median follow-up for patients still living is 95 months. Proteins from 30 mg of tissue were extracted in 300 μl 5% SDS at 90° C. for 5 minutes. Protein concentration was determined using the BCA method (Pierce, Rockford, Ill.); typical yields were 2-5 μg/μl. Samples were stored at −70° C. until use.

### Example 10

### Cell Line Standard

MCF-7 breast cancer cells were grown in 100 mm tissue culture dishes. Harvesting and extraction were a single-step process in the culture dish in which the cells were washed twice with cold phosphate-buffered saline, and proteins were then extracted with 5% SDS in distilled water at 90° C. for 5 minutes. The extraction mixture was centrifuged, and protein remaining in the supernatant (yield about 1 μg/μl) was determined using the BCA method. This standard preparation was stored at −70° C.

### Example 11

### Western Blot Assay for AIB1

Assays were performed and results quantified by an investigator blinded to clinical information. 20 μg of extracted protein in sample buffer (0.05 M tris, pH 6.8, containing 2% SDS, 2.5% β-mercaptoethanol, 10% glycerol, and 0.1% bromphenol blue as tracking dye) were placed in boiling water for 2 minutes, cooled to room temperature, and centrifuged for 1 minute. An MCF-7 reference standard (5 μg protein per lane) was included on each gel as a normalization control. Proteins were electrophoresed on 8% Tricine-Glycine polyacrylamide gels (Invitrogen, Carlsbad, Calif.) and then transferred onto nitrocellulose. Blots were rinsed 5 minutes with TBS (TRIS-buffered saline: 100 mM TRIS, pH 7.5, and 0.9% NaCl) containing 0.1% Tween-20 (TBST). After blocking with 5% nonfat dry milk in TBST, the blots were incubated overnight at 4° C. in the primary antibody (1:1000 rabbit anti-RAC3 antiserum). This antibody was raised in rabbits against a MBP fusion protein containing amino acid residues 582-842 of human RAC3 (AIB1). The antibody is specific for AIB1 and does not cross-react with other members of the SRC family.

The blots were washed 3 times in TBST, followed by incubation for 1 hour in 5% nonfat dry milk in TBST and 1:5000 horseradish peroxidase labeled anti-rabbit Ig (Amersham, Piscataway, N.J.). The blots were washed 5 times in TBST, after which the labeled protein was visualized on a FluorChem digital imaging system (Alpha Innotech, San Leandro, Calif.) using an enhanced chemiluminescence detection system. Band intensities were measured densitometrically using the AlphaEaseFC software (Alpha Innotech, San Leandro, Calif.). AIB1 levels in tumors were normalized to the AIB1 concentration in the MCF-7 positive control lysate (5 μg) from the same immunoblot.

### Example 12

### Analytical Methods

ER and progesterone receptors (PR) were measured by ligand binding assays as described by Harvey et al., 1999. S-phase fraction was calculated by DNA flow cytometry and classified as low, intermediate, or high (Wenger et al, 1998). Immunohistochemistry was used to semiquantitatively measure p53 and bcl 2 expression, and HER-2 expression was determined by western blot analyses as described by Elledge et al., 1997, and Tandon et al., 1989.

### Example 13

### Statistics

Associations between AIB1, steroid receptors, HER-2 and other molecular markers, and clinical variables were assessed using Spearman rank correlation. The disease-free survival (DFS) was calculated from the date of diagnosis. First recurrence (local or distant) was scored as an event, and patients without recurrence were censored at the time of last follow-up or death, meaning that an event of interest (e.g., recurrence or death) has not yet occurred. For graphical presentation, follow-up was truncated at 120 months. Overall survival was calculated from the date of diagnosis, with death from any cause being scored as an event. Patients who were alive at the last follow-up were censored at the last follow-up date. Survival curves were derived from Kaplan-Meier estimates, which are well known in the art, and the curves were compared by log-rank tests. Initially, separate analyses were performed for treated and untreated patients. The influence of AIB1 adjusted for other prognostic factors was assessed in multivariate analysis by Cox proportional hazards models.

All statistical tests were two-sided at the 5% level of significance, and were performed using SAS Version 8.0. Survival rates and hazard ratios are presented with their 95% confidence intervals (CI).

### Example 14

### Chemicals and Antibodies

EGF and heregulin (HRG) were purchased from Invitrogen (Carlsbad, Calif.) and R & D System (Minneapolis, Minn.), respectively. Both were dissolved in sterile distilled water according to the manufacturer's instructions. 17 Beta-estradiol (E2), 4-hydroxy tamoxifen (4HOT) and all other chemicals were from Sigma (St. Louis, Mo.) except where indicated. Polyclonal phospho-ER antibody (Serine 118), which specifically recognized human ER alpha only when phosphorylation at residual serine 118 was described previously ^{(Ali,S)}. Antibody against total ER was purchased from NeoMarker (Fremont, Calif.). Polyclonal antibodies against total and phospho-EGFR (at site 845), HER-2 (at site 1248), non-phospho and phospho Akt (Ser437) and p-ERK1, 2 MAPK (T202/Y204) from Cell Signaling Technology (Berkeley, Mass.). β-actin antibody from Chemicon (Temecula, Calif.).

### Example 15

### Cell Lines and Treatment

MCF-7 and the derivative MCF-7 HER-218 cell line were maintained as known in the art. Both cell lines have high levels of AIB1 expression due to gene amplification. The MCF-7 HER-218 cell line overexpresses HER-2. See Brooks et al., 1973. Treatments of cells with E2, 4HOT, EGF or HRG were conducted after cells were starved in phenol red-free, serum-free IMEM for 24 hr.

### Example 16

### Phosphorylation Analysis, Preparation of Cell Extraction and Western Immunoblotting

Treatment with estrogen (1 nM, 20 min) or tamoxifen (100 nM, 20 min), or epidermal growth factor (100 ng/ml, 10 min), or heregulin (10 ng/ml, 20 min). Treated cells were then immediately harvested. Cells were first rinsed twice with cold PBS, and lysed in 1×cell lysis buffer (Cell Signaling Technology) supplemented with 10% Glycerol, 1 mM Pherryl Methylsulfonyl Fluoride (PMSF), 1×Complete Protease inhibitor mixture (Roche, Indianapolis, Ind.), 1 μM Okadaic acid, 10 μg/mL Microsystin as instruction from Cell Signaling Technology. Protein concentration was measured by Bio-Rad Protein Assay kit (Bio-Rad, Hercules, Calif.) according to manufacturer's instruction. 20 μg of protein extracts from individual treatments were electrophoresized on SDS-PAGE gel (Invitrogen), and electroblotted to nitrocellulose membrane (Millipore, Bedford, Mass.). Membranes were blocked with blocking buffer (5% w/v nonfat dry milk in 1×TBS with 0.1% Tween-20, TBST) for 1 hr and then incubated with primary antibody in 5% BSA TBST (for all phospho antibodies with gentle agitation overnight at 4° C., otherwise in blocking buffer for 2 hrs at room temperature). After washing and incubating with secondary horseradish peroxidase conjugated antibodies against mouse or rabbit IgG (Amershan, Arlington Heoghts, Ill. Immunoreactive proteins were detected by the ECL (Amershan). Quantitation was performed by using NIH Imagine Software.

### Example 17

### Western Blot Analysis Showing Mobility Shift of AIB1

Whole cell extractions of 10 μg, from serum-starved (o/n) MCF7 or MCF7/HER-218 cells treated with either E2 (10⁻⁹ M, 20 min), or 4-hydroxy-tamoxifen (10⁻⁷ M, 20′), HRG (10 ng/ml, 20 min), (1 μM, 3 hrs), were electrophoresised in 6% SDS-PAGE gel. In PPase treatment group, cell extracts were incubated with PPase prior to analysis as previously described. Antibody against AIB1 was used.

### Example 18

### Transient Transfection and ER Dependent Transcription

Cells were maintained in 10% Dextran-coated charcoal-stripped fetal calf serum (DCCS FCS) in phenol red-free IMEM for one week as described as above. MCF-7/HER-218 cells were plated 24 hrs before starvation in phenol red-free, serum-free IMEM for 24 hrs. For purpose of consistency of transfection efficiency, cells were cotransfected 0.5 μg luciferase reporter construct containing 3×ERE with 0.4 ng beta-galactosidase construct containing TK promoter per 1×10⁵ cells in 1% DCCS FCS, phenol red-free Opti-MEM I medium (Invitrogen) using LipofectAmine (Invitrogen) according to manufacturer's instruction. Cells were splited into various treatment groups at density of 1×10⁵ cells per well (12 wells plate) in 1% DCCS FCS, phenol red-free Opti-MEM I medium 12 hrs after transfection. After 2 hours, medium in each well was replenished with fresh 1% DCCS FCS, phenol red-free IMEM with or without E2 or 4HOT in the absence of heregulin and cells were continue to incubate for another 16 hrs. Luciferase activity in cell lysate was determined by using Luciferase Assay System (Promega, Madison, Wis.) according to manufacturer's instruction in Luminometer (Thermo Labsystems, Helsinki, Finland). Beta-galactosidase activity was measured after reaction of certain amount of lysate with Chlorophenol red-beta-D-galactopyranoside (CPRG, Roche, Indianapolis, Ind.) by Optical Density as described previously (29). Luciferase activity was normalized by beta-galactosidase activity.

### Example 19

### Soft-Agarose Colony Formation Assay

MCF7/HER-218 cells used for experiment were maintained in 10% Dextran-coated charcoal-stripped fetal calf serum (FBS, Hyclone) in phenol red-free IMEM for one week, exponential growing cells were used for soft-agarose colony assay. 5×10³ of MCF-7 or MCF7/HER-218 cells were suspended in 1 ml of top soft agar containing 0.33% agarose, 10% Dextran-coated charcoal-stripped FBS in phenol red-free IMEM at 37° C. without estrogen or tamoxifen which was for pretreatment, which placed on top of solidified support lay containing 1% agarose and 10% Dextran-coated charcoal-stripped FCS in phenol red-free IMEM at 37° C. at bottom of each well of six-well plate. The plates were incubated for three weeks in humidified 5% CO₂-95% air incubator at 37° C. Formation of colony was examined under dissection microscope. Clusters more than 50 μm diameter were defined and counted as colonies.

### Example 20

### Chromatin Immunoprecipitation Assays

Wild type MCF7 and HER-2-18 cells were grown to 90% confluence in phenol red-free IMEM supplemented with 10% CDS FBS for 7 days, the cells were starved completely with phenol red-free IMEM (without serum) for overnight. Starved cells then were treated estrogen (1 nM) or tamoxifen (100 nM) for 20 min, immediately followed by crosslinking, where 1% formaldehyde (final concentration) was directly added to medium and kept at 37 C. for 10 min. Cells were washed with cold PBS containing protease inhibitor cocktail (Roche) and 1 mM PMSF, and collected into tube by scraping. Cells were pelleted and lysis in 1 ML SDS lysis buffer and incubated on ice for 10 min. Lysate was sonicated by three times for 10 sec. at maximum setting followed by centrifugation for 10 min at 4 C. 200 ul of supernatant was, subjected to each immunoprecipitation with specific antibody, diluted with ChIP dilution buffer 10 fold and pre-cleared with 80 ul of SS-DNA/Protein A/G agarose plus 20 ul of normal mouse or rabbit IgG (Santa Cruz) depending on primary antibody used afterward for 4 hrs at 4 C. The beads were collected and saved as normal IgG control. The pre-cleared supernatant was immunoprecipitated with antibodies against ER, or AIB1, or NcoR, or Act-H3, respectively at 4° C. for overnight, followed by SS-DNA/Protein A/G at 4 C. for 1 hr. Pellet agarose by gentle centrifugation (1000 rpm, 1 min, 4° C.). The pellets were intensively washed sequentially as follows in 1 ml volume: low salt wash buffer X 1 time, high salt wash buffer X 1 time, LiCl salt wash buffer X 1 time, TE X 2 times. Immunocomplex beads were eluted by 250 ul elution buffer for 15 min at room temperature for 2 tims, 20 ul 5 M NaCl were added to pooled elution solution and incubated at 65° C. for 4 hrs. DNA fragments in crosslinking reversed solution were purified by using Qiagene Gel Extraction Kit. For PCR, 5 ul from 30 ul DNA elution was used. PCR products were separated in 2% agarose gel.

### Example 21

### Cell Treatment for Endogenous Gene Induction

Cells in 10 cm diameter were grown to 90% and completely starved in serum-free, phenol red-free IMEM for overnight and then were induced by estradial (1 nM), or 4-hydroxy-tamoxifen (100 nM), or heregulin (10 ng) for 12 hr. The cellharvest, cell extraction preparation and immunobloting were as above.

### Example 22

### HER-2 Overexpression Results in Tamoxifen-Stimulated Growth of Breast Cancer Cells

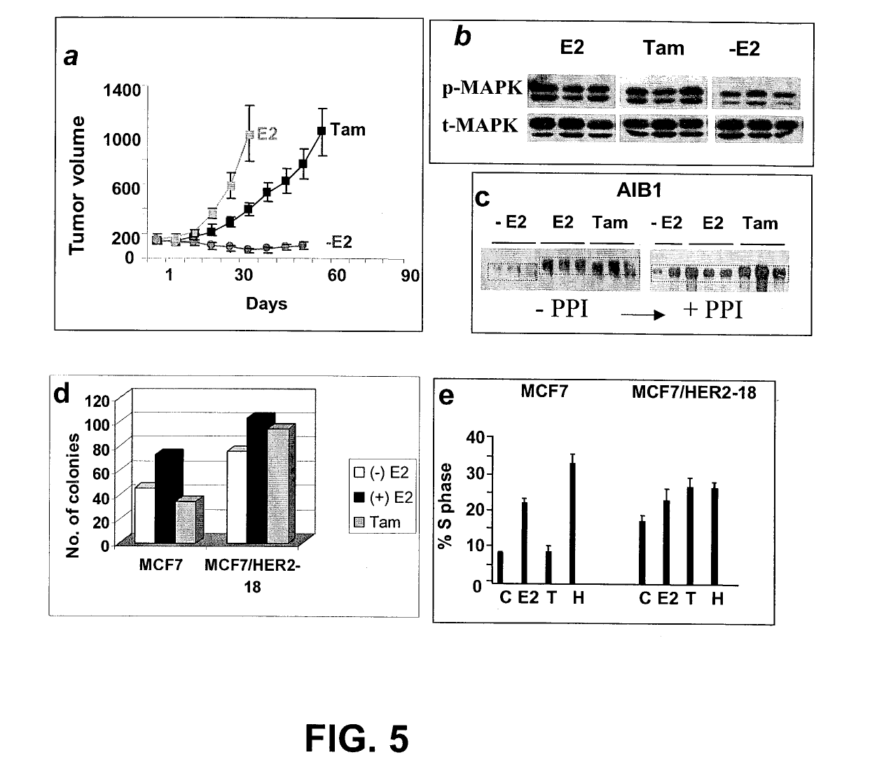

A group of mice with small MCF-7/HER-218 tumors with continued estrogen, estrogen withdrawal alone, or estrogen withdrawal combined with tamoxifen (FIG. 5A). Tumor growth was stimulated by estrogen and strikingly inhibited by estrogen withdrawal, indicating continued dependence of these tumors on estrogen for growth rather than estrogen “independence”. Tumor growth was also stimulated by treatment with tamoxifen, in contrast to wild-type MCF-7 cells which are inhibited by the drug. Tumors grew somewhat more slowly with tamoxifen than with estrogen, but their growth was reproducibly enhanced compared to estrogen withdrawal alone in multiple experiments. These data indicate that tamoxifen, like estrogen, functions as an agonist to enhance tumor growth when HER-2/neu is overexpressed in these cells. Interestingly, the level of phosphorylated (active) MAPK was also altered by the three hormonal treatments (FIG. 5B). Tumor levels were high in mice treated with estrogen and in those treated by estrogen withdrawal plus tamoxifen, while they were much lower in mice treated with estrogen withdrawal alone. This result indicate that estrogen and tamoxifen activate MAPK.

### Example 23

### Crosstalk Between Growth Factor Receptor and ER Pathways

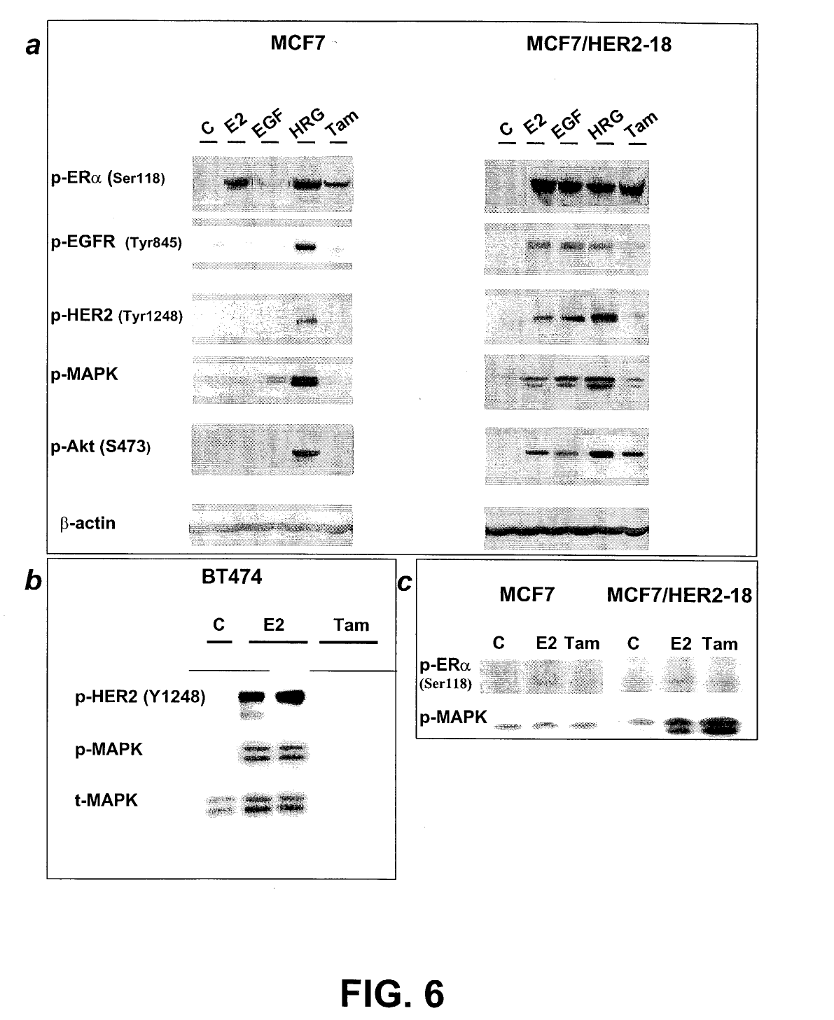

To further investigate the mechanism by which the estrogen agonist properties of tamoxifen-bound ER are increased when HER-2/neu is overexpressed, we examined the short-term (20 minutes) effects of estrogen, EGF, heregulin, and tamoxifen, to block growth factor signaling, on phosphorylation of ER, EGF-R, HER-2/neu, MAPK and AKT in the parental MCF-7 and in the MCF-7/HER-218 cells. FIG. 6A demonstrates by immunoblotting that both estrogen and tamoxifen phosphorylate the estrogen receptor on serine 118,. EGF and heregulin, the latter which binds to HER-3 and activates EGF-R and HER-2/neu via heterodimerizatio, also phosphorylate ER on serine 118,. We were unable to detect phosphorylation of EGF-R on tyrosine 845 or HER-2/neu on tyrosine 1248 with EGF in these cells which express low levels of these receptors, although EGF did phosphorylate MAPK and AKT indicating that EGF-R is functional in these cells (FIG. 6A). Similar results were obtained using other antibodies specific for tyrosine 992, 1048, and 1068 on EGF-R and 887 and 1112 on HER-2/neu. Heregulin, on the other hand, strongly phosphorylated ER, the EGF-R, HER-2/neu, AKT and MAPK. While EGF and heregulin activated ER in these cells, we could not detect phosphorylation of EGF-R, HER-2/neu, MAPK, or AKT by estrogen or tamoxifen indicating receptor crosstalk in only one direction in the parental MCF-7 cells or that the phosphorylated proteins were below the limit of detection in our assay.

In contrast, bidirectional crosstalk was easily detected in the MCF-7/HER-218 cells (FIG. 6A). Similar to the parental cells, estrogen, EGF, heregulin and tamoxifen all phosphorylated the ER on serine 118. Importantly, in contrast to the parental MCF-7 cells, phosphorylation was detected of EGF-R, HER-2/neu and MAPK by EGF and heregulin but also by both estrogen and tamoxifen in the MCF-7/HER-218 cells. Therefore, phosphorylation of these growth factor signaling molecules by estrogen and tamoxifen is totally dependent on the receptor tyrosine kinase pathway since it was not evident absent overexpression of HER-2.

To confirm the effects of estrogen and tamoxifen on HER-2/neu signaling in another cell line, BT474 cells were used. These cells express ER and they are naturally amplified for the HER-2/neu gene. A striking increase in phosphorylated HER-2/neu and MAPK was observed with both estrogen and tamoxifen in these cells (FIG. 6B).

All of the experiments in FIGS. 6A and 6B involved a 20 minute treatment with estrogen and tamoxifen. Similar effects were observed at time points as early as 5 minutes. We also examined the effects of a 48 hour treatment with estrogen and tamoxifen to determine if the observed effects were only transient (FIG. 6C). In wild-type MCF-7 and in the MCF-7/HER-218 cells phosphorylation of ER by estrogen and tamoxifen was still evident at the longer time point. Again no effect of estrogen or tamoxifen on MAPK phosphorylation was evident in the MCF-7 cells, while striking effects were seen in the MCF-7/HER-218 cells. Thus, in these HER-2/neu overexpressing cells, estrogen and tamoxifen activate growth factor signaling while at the same time growth factor signaling activates ER. Inhibition of the EGF-R/HER-2 tyrosine kinases blocks the crosstalk in both directions.

### Example 24

### Phosphorylation of AIB1

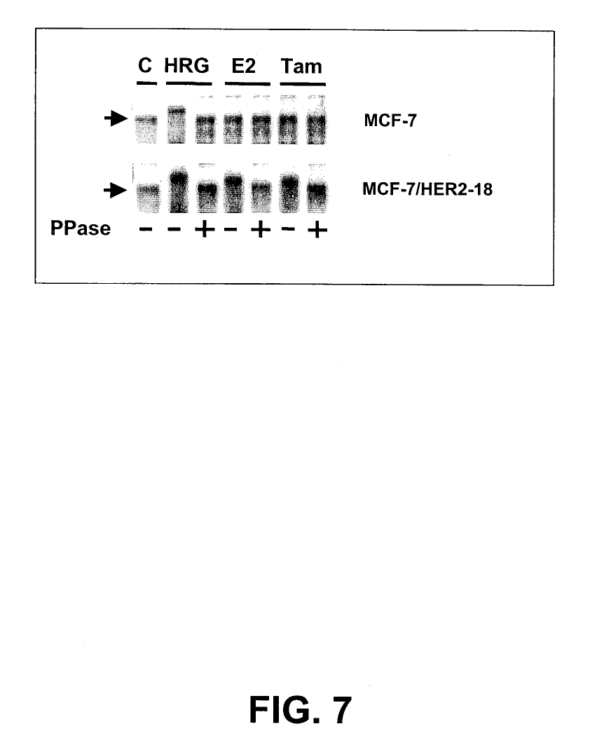

In our prior study in tumors from patients treated with tamoxifen, high levels of expression of both AIB1 and HER-2/neu were required for the tamoxifen resistant phenotype. Since MAPK phosphorylates AIB1, we reasoned that tamoxifen resistance in such patients is mediated in part by the functional activation of AIB1 by HER-2 receptor signaling, an affect that would strongly enhance the agonist properties of tamoxifen-bound ER. Specific antibodies to phosphorylated forms of AIB1 are not yet available so we used mobility shifts of the specific AIB1 band on immunoblots with or without phosphatase treatment to evaluate the effects of heregulin, estrogen and tamoxifen on AIB1 phosphorylation in the MCF-7 and MCF-7/HER-218 cells (FIG. 7). In the wild-type cells, only heregulin upshifted the AIB1 band, an effect that was reversed by phosphatase treatment indicating that the retarded band was indeed a phosphorylated form of the protein. In contrast, in the MCF-7/HER-218 cells phosphorylation of AIB1 was observed not only with heregulin, but also with estrogen and tamoxifen, indicating that this AIB1 phosphorylation was due to ER-mediated activation of the EGF-R/HER-2/neu pathway. These data indicate that the growth stimulatory properties of tamoxifen in the MCF-7/HER-218 cells result both from its activation of growth factor signaling and, indirectly, from the downstream activation of AIB1 which would enhance its agonist properties on ER. An inhibitor of the HER-2/EGFR pathway will block the bidirectional crosstalk in these cells and should also restore tamoxifen's antagonist properties on gene expression and tumor growth, and thereby overcome tamoxifen resistance.

### Example 25

### Agonist Activity of Tamoxifen on ER Dependent Gene Transcription in HER-2 Overexpressing Breast Cancer Cells

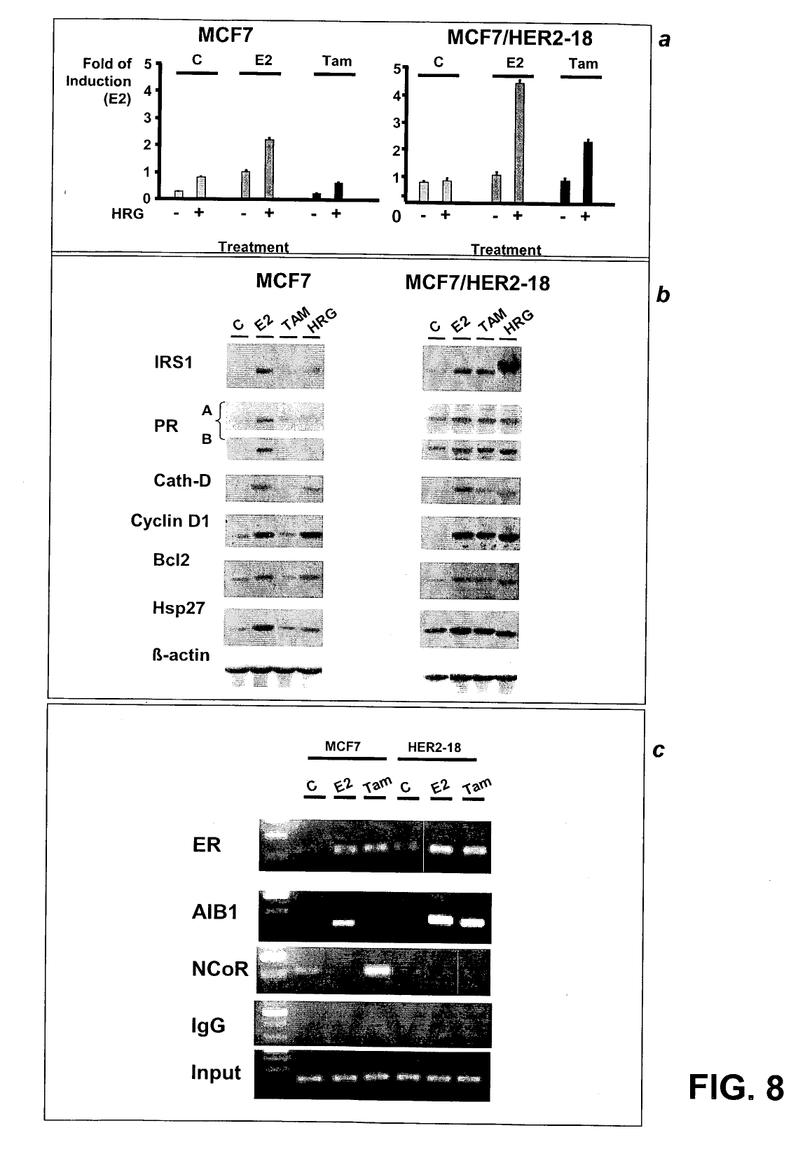

FIG. 8A contrasts the effects of estrogen, tamoxifen, and heregulin in the MCF-7 and the MCF-7/HER-218 cells transiently transfected with an ERE luciferase reporter gene as a marker of ER-dependent gene transcription. The luciferase activity observed with estrogen treatment was set to one for both cell lines and the activity observed with the other treatments expressed as fold induction. In the parental MCF-7 cells ER dependent transcription is induced by estrogen. The addition of heregulin to estrogen, however, further increased luciferase activity two fold, an effect that is growth factor receptor dependent. As expected, tamoxifen had no agonist activity in these cells, but the addition of heregulin to tamoxifen did increase luciferase activity a small amount.

In the MCF-7/HER-218 cells, however, baseline luciferase activity was increased dramatically (FIG. 8A). The addition of heregulin to these cells remarkably enhanced luciferase activity in response to both estrogen and tamoxifen. The inability of estrogen or tamoxifen alone to increase luciferase activity above control may be due to enhanced sensitivity to the low residual estrogen of the activated ER in these cells which significantly raised the baseline luciferase activity. Importantly, heregulin further boosted the estrogen agonist properties of tamoxifen to levels above estrogen alone. Thus, tamoxifen functions as an estrogen agonist on ER dependent gene transcription when HER-2/neu is overexpressed and activated.

To confirm the agonist properties of tamoxifen on gene expression in a more physiological context in cells overexpressing HER-2/neu, a panel of endogenous estrogen responsive genes with various promoter types was examined by comparing protein expression on immunoblots (FIG. 8B). In wild-type MCF-7 cells estrogen variably increased the expression of all of the genes examined. As expected, tamoxifen demonstrated no agonist activity on any of these genes. Interestingly, heregulin by itself induced the expression of all of the genes except for progesterone receptor. Whether the effects of heregulin on protein expression are mediated through its activation of ER or by some other mechanism remains to be defined.

Estrogen had similar effects in the MCF-7/HER-218 cells (FIG. 8B). However, in contrast to wild-type cells, in the MCF-7/HER-218 cells tamoxifen behaved as an estrogen agonist on all of the genes including those thought to be important for cell proliferation and/or survival, such as IRS1 and cyclin-D1. This agonist activity of tamoxifen was totally dependent on growth factor receptor activity. Heregulin also had dramatic effects on several of these genes. There was a large increase in the level of IRS1 and the band migrated more slowly indicating that heregulin treatment also caused phosphorylation of the protein. Heregulin even increased the protein expression of progesterone receptor. Thus, in the setting of HER-2/neu overexpression, in MCF-7/HER-218 cells tamoxifen behaves as an estrogen agonist on a variety of estrogen dependent genes with various response elements in the promoter.

Having shown that tamoxifen has estrogen agonist activity on estrogen target genes in the presence of enhanced HER-2/neu signaling, the assembly of ER transcription complex components in the MCF-7 cells compared to the MCF-7/HER-218 was studied cells using the pS2 promoter (FIG. 8C). In normal cells and in wild-type MCF-7 cells, estrogen preferentially recruits coactivators in the P160 family like AIB1 while tamoxifen preferentially recruits corepressors such as N-CoR. The endogenous transcription complexes present on the pS2 promoter was studied using chromatin immunoprecipitation. After a 20 minute incubation with estrogen or tamoxifen, the presence of the pS2 promoter in the chromatin immunoprecipitates was analyzed by semi-quantitative PCR. In the MCF-7 cells, estrogen induced occupancy of the pS2 promoter by both ER and AIB1. Tamoxifen also induced occupancy of this promoter by ER but not by AIB1. Instead, the corepressor N-CoR was present in the complex. However, in the MCF-7/HER-218 cells both estrogen and tamoxifen induced occupancy of the pS2 promoter by ER and by AIB1, a process that is largely largely growth factor dependent. The presence of ER and AIB1 on the promoter when the receptor is bound by tamoxifen explains why tamoxifen behaves as an agonist in these cells. Therefore, in distinct contrast to the agonist effects of estrogen, those of tamoxifen were completely dependent on crosstalk with the EGF-R/HER-2/neu pathway since they were nearly abolished by the receptor tyrosine kinase inhibitor.

### Example 26

### Athymic Nude Mouse Model

Ovariectomised female BALB/c-nu+/nu+ mice 4-5 weeks of age were purchased from Harlan Sprague-Dawley (Madison, Wis.). Methods for housing and maintenance of the animals were in accordance to institutional guidelines and were previously described. Estrogen pellets (0.25 mg, 21 day release) were purchased from Innovative Research (Rockville, Md.) and half a pellet placed subcutaneously in the interscapular area of each animal on the day prior to cell injection. MCF-7/HER-218 cells were grown in 150 cm³ flasks and split 1:5 in DMEM (Invitrogen, Calif.) supplemented with 10% FBS. When flask cell confluence reached 90-100%, cells were harvested using a soft rubber scraper, then spun down at 1000 rpm for 3 minutes, and resuspended in the original media for injection. Cells were injected at 5×10⁶−1×10⁷ in 0.2 ml per animal in the right mammary fat pad. When tumors reached a size of 100-200 mm³ they were randomly assigned to continue estrogen supplementation, estrogen deprivation by surgical pellet withdrawal, or to estrogen deprivation plus 5 days a week tamoxifen injection at 500 μg/animal per day. Animals in each one of these groups were either given concomitant Herceptin (10 mg/kg) or control vehicle. Each individual group had 8 animals (n=8). Herceptin or vehicle were given intraperitoneally at a dose of 10/mg/kg twice a week.. Tumors were subsequently measured weekly and recorded according to the formula: Tumor volume=width²×length/2. Animal were weighed every 2 weeks and monitored for toxicity. Tumor growth curves were constructed using the mean of tumors' volume at each measurement point with the error bars representing the standard error of the mean. Student's t-test was used to compare data between two groups. Values are expressed as relative to tumor volume to size at the beginning of treatment as mean ±s.d.

### Example 27

### Herceptin Restores Tamoxifen's Antagonist Activity on Anchorage Independent Colony Growth In Vitro and Tumor Growth In Vivo

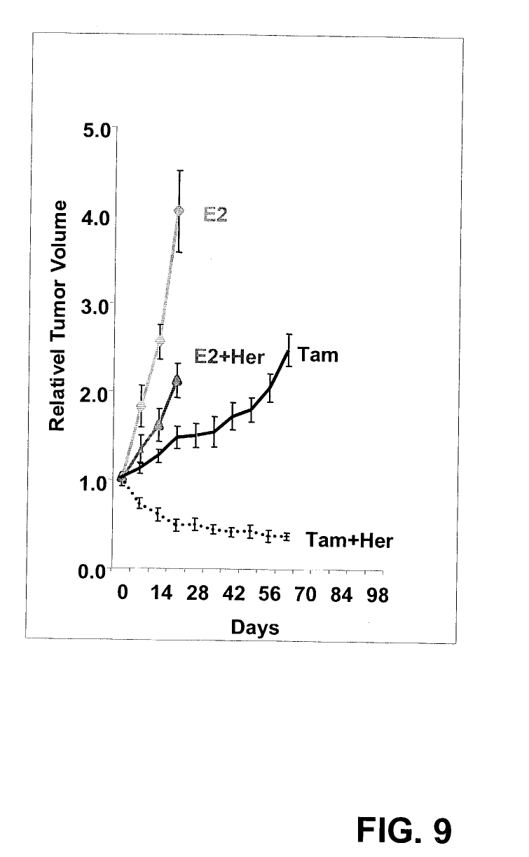

Herceptin blocks EGF-R/HER-2/neu crosstalk with ER, dissociates AIB1 from tamoxifen-liganded ER complexes on the promoter of target genes, and restores tamoxifen's antagonist effects on gene expression. FIG. 9 shows the results of an anchorage independent growth assay using the parental MCF-7 cells compared to the MCF-7/HER-218 cells. In MCF-7 cells estrogen increased colony formation above control, and Herceptin at concentrations of 1 μM or less, had no effect. Tamoxifen, as expected, inhibited colony formation in these cells, and, interestingly, its antagonist properties were reproducibly further enhanced by Herceptin. This finding indicates that even in cells with low levels of EGF-R/HER-2/neu, tamoxifen still has weak agonist activity that is growth factor dependent and that can be reduced by inhibiting the receptor tyrosine kinases.

Like estrogen, tamoxifen stimulated MCF-7/HER-218 colony formation (FIG. 9). This agonist activity of tamoxifen is entirely dependent on growth factor signaling since its antagonist activity was totally restored with Herceptin. Estrogen-induced colony formation was also partially growth factor-dependent in these cells. Estrogen still stimulated colony growth above control, but Herceptin reduced both estrogen-induced and control colony growth.

To confirm the agonist effects of tamoxifen on cell proliferation in the MCF-7/HER-218 cells, the S-phase fraction of cells was measured after treatment with estrogen, tamoxifen or heregulin with and without Herceptin. In the MCF-7 cells, estrogen treatment increased the percent of cells in S-phase, while tamoxifen had no agonist effect (FIG. 9). Heregulin also markedly increased the S-phase fraction and this effect was blocked by Herceptin. In contrast, MCF-7/HER-218 cells growing under control conditions had a significantly higher basal S-phase fraction that was reduced by Herceptin. In these cells tamoxifen, like estrogen and heregulin, also stimulated the fraction of cells in S-phase. Herceptin abrogated the stimulatory effect of tamoxifen and heregulin, while it only modestly inhibited estrogen's effects.

To determine if tamoxifen-stimulated growth of MCF-7/HER-218 tumors is also growth factor dependent in vivo, randomized estrogen-supplemented athymic mice with small tumors were subjected to continued estrogen or to estrogen deprivation plus tamoxifen without or with Herceptin (FIG. 9). In contrast to parental MCF-7 tumors in which estrogen-mediated growth is unaffected by Herceptin, the receptor tyrosine kinase inhibitor did slow estrogen-induced growth of MCF-7/HER-218 tumors slightly compared to estrogen treatment alone. In contrast, the agonist properties of tamoxifen on tumor growth were totally growth factor dependent and were abolished by concomitant treatment with Herceptin. Finally, the effects of these treatments on the phosphorylation state of AIB1 were examined(FIG. 9). Estrogen treatment was associated with both higher levels of AIB1 and a more slowly migrating band on the immunoblot. Likewise, total and phosphorylated AIB1 were both higher in the tamoxifen-treated tumors consistent with its estrogen agonist qualities. The specificity of the phosphorylated bands is proven by pretreatment of the extracts with phosphatases.

### Example 28

### Mechanism of Tamoxifen resistance

The observation between high levels of AIB1 correlating to a more favorable disease-free survival only in patients not receiving adjuvant therapy is an unexpected result. Breast cancers arising in women taking estrogen replacement therapy are more indolent and are associated with more favorable patient outcome. Enhanced signaling through ER in women taking exogenous estrogen promotes and maintains a more differentiated and less biologically aggressive tumor. High AIB1 levels, by further augmenting ER agonist activity, then promote a tumor with more indolent behavior and with reduced metastatic potential, but also one that is less responsive to tamoxifen. Thus, high expression of ER coactivators reduce the estrogen antagonist activity of tamoxifen-bound receptor in breast cancer patients and, consequently, determining AIB1 levels is an important predictive marker for tamoxifen resistance in clinical breast cancer. Further, high levels of AIB1 must be present for the tamoxifen resistance associated with HER-2 overexpression to be manifest clinically.

A mechanism for this resistance is presented in ER-positive breast cancer cells that express high levels of AIB1 and HER-2/neu. The agonist activity of tamoxifen on gene expression and cell proliferation is strikingly increased in these cells resulting in tumor growth stimulation by the drug. These effects are in stark contrast to tamoxifen's antagonist effects in the same parental cells with low HER-2/neu expression, and they closely mimic those from the clinical study of tumors from tamoxifen-treated patients described above. Additionally, an explanation for tamoxifen's estrogen-like activity in these cells is provided. Enhanced signaling from the EGFR and HER-2/neu tyrosine kinases activates MAPK which then phosphorylates and functionally activates both ER and AIB1, confirming prior reports. In addition, both estrogen and tamoxifen rapidly activate EGFR, HER-2/neu, AKT and MAPK in these cells, thereby establishing bidirectional crosstalk and a vicious cycle of cell survival and proliferative stimuli even when ER is bound by tamoxifen. This crosstalk is not evident and tamoxifen remains an antagonist in cells with low growth factor receptor levels. Tamoxifen, like estrogen, induces phosphorylation of ER on serine 118 even in the parental cells in which it is an antiestrogen. This clearly indicates that phosphorylation of ER on serine 118 alone does not increase the agonist effects of tamoxifen sufficiently to cause resistance. In contrast to other cell types and to parental MCF-7 cells, in the HER-2/neu overexpressing cells both estrogen and tamoxifen recruit ER and AIB1 to the promoter of an estrogen target gene as determined by chromatin immunoprecipitation assays. Instead, in the parental cells, tamoxifen-bound ER recruits the corepressor N-CoR to the promoter complex. In the HER-2/neu overexpressing cells the receptor tyrosine kinase inhibitor blocks phosphorylation of AIB1 and its recruitment to the PS2 promoter, allowing the corepressor N-CoR to interact with ER on the promoter. The presence of AIB1, which recruits other proteins with histone acetyltransferase activity and which would enhance gene transcription, explains why tamoxifen behaves as an estrogen agonist on a panel of endogenous target genes, and as an agonist on in vitro and in vivo proliferation of the MCF-7/HER-218 cells. Thus, in these cells the classical actions of ER on transcription are enhanced by its nongenomic effects to activate the growth factor receptor tyrosine kinases in response to estrogen or tamoxifen.

Low levels of ER and no AIB1 were detected on the pS2 promoter in the absence of estrogen or tamoxifen in the HER-2/neu overexpressing cells. Furthermore, in tumors from mice treated by estrogen withdrawal alone, MAPK levels fell markedly and phosphorylation of AIB1 was no longer detected. Finally, estrogen withdrawal was a strikingly effective inhibitor of in vivo growth of these cells, indicating that ovariectomy in premenopausal women and aromatase inhibition in postmenopausal women may still be worthwhile therapies in tumors overexpressing EGFR/HER-2/neu and /or AIB1.

Simultaneous treatment with a receptor tyrosine kinase inhibitor, similar to treatment with estrogen withdrawal, blocks the crosstalk between the ER and growth factor receptor pathways. The inhibitor would prevents activation of ER and AIB1, reduces the recruitment of AIB1 and enhances recruitment of N-CoR to tamoxifen-bound ER complexes on promoters of target genes, and then restores tamoxifen's antagonist effects on gene expression and tumor growth.

### References

All patents and publications mentioned in the specification are indicative of the level of those skilled in the art to which the invention pertains. All patents and publications are herein incorporated by reference to the same extent as if each individual publication was specifically and individually indicated to be incorporated by reference.

Patents

U.S. Pat. No. 3,817,837

U.S. Pat. No. 3,850,752

U.S. Pat. No. 3,939,350

U.S. Pat. No. 3,996,345

U.S. Pat. No. 4,277,437

U.S. Pat. No. 4,275,149

U.S. Pat. No. 4,366,241

U.S. Pat. No. 4,608,251

U.S. Pat. No. 4,601,903

U.S. Pat. No. 4,599,231

U.S. Pat. No. 4,599,230

U.S. Pat. No. 4,596,792

U.S. Pat. No. 4,578,770

U.S. Pat. No. 4,554,101

U.S. Pat. No. 4,682,195

U.S. Pat. No. 4,472,509

U.S. Pat. No. 4,879,236

U.S. Pat. No. 4,938,948

U.S. Pat. No. 5,168,053

U.S. Pat. No. 5,196,066

U.S. Pat. No. 5,354,855

U.S. Pat. No. 5,624,824

U.S. Pat. No. 5,625,047

U.S. Pat. No. 5,645,897

U.S. Pat. No. 5,705,629

U.S. Pat. No. 5,871,986

WO 98/57982

WO 84/03564

EP 266,032

Non-Patented Literature

Bangham et al., “Diffusion of univalent ions across the lamellae of swollen phospholipids,” J. Mol. Biol., 13:238, 1965.

Brooks et al., “Estrogen receptor in a human cell line from breast carcinoma,” J. Biol. Chem., 248(17):6251-3, 1973.

Deamer et al., “Liposome Preparation: Methods and Mechanisms,” LIPOSOMES, M. Ostro ed. (1983).

Ghosh et al., “Targeting of liposomes to hepatocytes,” In: Wu G. Wu C ed., Liver diseases, targeted diagnosis and therapy using specific receptors and ligands, New York: Marel Dekker, pp. 87-104, 1991.

Gregoriadis, DRUG CARRIERS IN BIOLOGY AND MEDICINE, G. Gregoriadis (ed.), 1979, pp. 287-341.

Harlow et al., “Case-control study of borderline ovarian tumors: reproductive history and exposure to exogenous female hormones,” Cancer Res. 1988 October 15;48(20):5849-52.

Horwitz et al., “Nuclear receptor coactivators and corepressors,” Mol. Endocrinol. 10: 1167-1177; 1996.

Kaneda et al., “Increased expression of DNA cointroduced with nuclear protein in adult rat liver,” Science, 243:375-378, 1989.

Kato et al., “Expression of hepatitis B virus surface antigen in adult rat liver,” J. Biol. Chem., 266:3361-3364, 1991.

Nicolau et al., “Liposomes as carriers for in vivo gene transfer and expression,” Methods Enzymol., 149:157-176, 1987.

Perlmann et al., “Nuclear receptors in Sicily: all in the famiglia,” Cell 90: 391-397

Reiter et al., “An isoform of the coactivator AIB1 that increases hormone and growth factor sensitivity is overexpressed in breast cancer,” J. Biol. Chem. 276:39736-39741, 2001.

Shibata et al., “Role of co-activators and co-repressors in the mechanism of steroid/thyroid receptor action” Recent. Prog. Hormone Res. 52: 141-165; 1997.

Szoka and Papahadjopoulos, “Procedure for preparation of liposomes with large internal aqueous space and high capture by reverse-phase evaporation,” Proc. Nat'l Acad. Sci. U.S.A. 75:4194-98 1978.

Tsai et al., “Molecular mechanisms of action of steroid/thyroid receptor superfamily members,” Ann. Rev. Biochem. 63: 451-486, 1994.

Wong et al., “Appearance of β-lactamase activity in animal cells upon liposome mediated gene transfer,” Gene, 10:87-94, 1980.

What is claimed is:

1. A method of identifying a patient with an endocrine therapy-resistant cancer comprising the steps of:
obtaining a sample from the patient; and
determining an AIB1 polypeptide level in the sample, wherein an elevated AIB1 polypeptide level as compared to a control sample indicates that the patient is endocrine therapy-resistant.

2. The method of claim 1, wherein the sample is a fluid, a tissue or a cell.

3. The method of claim 1, wherein the AIB1 polypeptide comprises an amino acid sequence substantially similar to SEQ ID NO:1.

4. The method of claim 1, wherein the cancer comprises an estrogen receptor-positive cancer or a progesterone receptor-positive cancer.

5. The method of claim 4, wherein the cancer is a breast, an ovarian, a prostate, or an endometrial cancer.

6. The method of claim 1, further comprising the step of determining a HER-2 polypeptide level in the sample, wherein an elevated HER-2 polypeptide level as compared to the control and the elevated AIB1 polypeptide level indicate that the patient is endocrine therapy-resistant.

7. The method of claim 1, wherein the AIB1 polypeptide comprises an amino acid sequence substantially similar to SEQ ID NO:1.

8. The method of claim 1, wherein the AIB1 polypeptide is an alternative splice variant of AIB1.

9. The method of claim 1, wherein the HER-2 polypeptide comprises an amino acid sequence substantially similar to SEQ ID NO:3.

10. The method of claim 1, wherein the endocrine therapy is tamoxifen, raloxifene, megestrol, or toremifene.

11. A method of providing a prognosis of disease-free survival in a cancer patient who is not receiving endocrine therapy comprising the steps of:
obtaining a sample from the patient; and
determining an AIB1 polypeptide level in the sample, wherein an elevated AIB1 polypeptide level as compared to a control sample indicates the prognosis of a high disease-free survival.

12. A method of providing a prognosis of disease-free survival in a cancer patient receiving an endocrine therapy comprising the steps of:
obtaining a sample from the patient; and
determining an AIB1 polypeptide level in the sample and a HER-2 polypeptide level in the sample, wherein an elevated AIB1 polypeptide level and an elevated HER-2 polypeptide level as compared to a control sample indicates the prognosis of a low disease-free survival.

13. A method of screening for a compound that improves the effectiveness of an endocrine therapy in a patient comprising the steps of:
introducing a test agent to a cell, wherein the cell comprises a polynucleotide encoding an AIB1 polypeptide operatively linked to a promoter; and
determining the AIB1 polypeptide level of the cell, wherein when the level is decreased following the introduction of the test agent, the test agent improves effectiveness of the endocrine therapy in the patient.

14. The method of claim 13, wherein the compound is a ribozyme, an antisense nucleotide, a promoter inhibitor, a kinase inhibitor, or a methyltransferase inhibitor.

15. A method of screening for a compound that improves the effectiveness of an endocrine therapy in a patient comprising the steps of:
contacting a test agent with an AIB1 polypeptide and an estrogen receptor (ER) polypeptide, wherein the AIB1 polypeptide or the ER polypeptide is linked to a marker, wherein the marker signaling is related to binding; and
determining the ability of the test agent to interfere with the binding of the AIB1 polypeptide and the ER polypeptide, wherein when the marker signaling is decreased following the contacting, the test agent improves effectiveness of endocrine therapy.

16. A method of identifying an antagonist of AIB1 comprising the steps of:
introducing a test agent to a cell, wherein the cell comprises a polynucleotide encoding an AIB1 polypeptide operatively linked to a promoter; and
determining the AIB1 polypeptide level of the cell, wherein when the AIB1 level is decreased following the introduction of the test agent, the test agent is an antagonist of AIB1.

17. A method of treating a cancer patient comprising the steps of:
introducing a test agent to a cell, wherein the cell comprises a polynucleotide encoding an AIB1 polypeptide operatively linked to a promoter; and
determining the AIB1 polypeptide level of the cell, wherein when the level is decreased following the introduction of the test agent, the test agent is an antagonist of AIB1; and
administering a therapeutically effective amount of the antagonist to the patient.

18. The method of claim 17, further comprising administering an endocrine therapy to the patient.

19. The method of claim 17, wherein the antagonist interferes with translation of the AIB1 polypeptide, interferes with an interaction between the AIB1 polypeptide and an estrogen receptor polypeptide, interferes with phosphorylation of the AIB1 polypeptide, or inhibits the function of a polypeptide encoding a kinase that specifically phosphorylates the AIB1 polypeptide.

20. A method of improving the effectiveness of an endocrine therapy in a cancer patient comprising administering a therapeutically effective amount of an antagonist of an AIB1 polypeptide to the patient to provide a therapeutic benefit to the patient.

21. A method for screening pre-menopausal women with breast cancer comprising the steps of:
obtaining a sample from the patient; and determining an AIB1 polypeptide level in the sample, wherein an elevated AIB1 polypeptide level indicates that ovariectomy is a treatment option.

22. The method of claim 21, further comprising determining a HER-2 polypeptide level.

23. A method of predicting de novo endocrine therapy resistance in a cancer patient comprising the steps of:
obtaining a sample from the patient; and
determining an AIB1 polypeptide level in the sample and a HER-2 polypeptide level in the sample, wherein an elevated AIB1 polypeptide level and an elevated HER-2 polypeptide level as compared to a control sample indicate de novo endocrine therapy resistance.
